(19) United States
(12) Patent Application Publication
(10) Pub. No.: US 2015/0197820 A1
(43) Pub. Date: Jul. 16, 2015

(54) MARKER SEQUENCES FOR INFLAMMATORY PROSTATE DISEASES, PROSTATE CARCINOMA AND THEIR USE

(71) Applicant: PROTAGEN AG, Dortmund (DE)

(72) Inventors: Helmut E. MEYER, Recklinghausen (DE); Angelika Lueking, Bochum (DE); Jens Beator, Kreiensen (DE); Axel Kowald, Bochum (DE); Georg Bartsch, Lans (AT); Helmut Klocker, Inzing (AT)

(21) Appl. No.: 14/672,334

(22) Filed: Mar. 30, 2015

Related U.S. Application Data
(63) Continuation of application No. 13/860,345, filed on Apr. 10, 2013.
Application No. 13/860,345 is a continuation of application No. 13/002,098, filed on Mar. 7, 2011, now abandoned.

(30) Foreign Application Priority Data
Jul. 4, 2008 (DE) 102008031699.7

(51) Int. Cl. C12Q 1/68

(52) U.S. Cl. CPC C12Q 1/6886; C12Q 2600/16; C12Q 2600/158

(57) ABSTRACT

The present invention relates to novel marker sequences for inflammatory prostate diseases, prostate carcinoma and the diagnostic use thereof together with a method for screening of potential active substances for inflammatory prostate diseases, prostate carcinoma by means of these marker sequences. Furthermore, the invention relates to a diagnostic device containing such marker sequences for inflammatory prostate diseases, prostate carcinoma, in particular a protein biochip and the use thereof.

### RELATED APPLICATIONS

This application is a continuation application of U.S. patent application Ser. No. 13/860,345, filed Apr. 10, 2013, which is incorporated by reference herein in its entirety, which was a continuation application of U.S. patent application Ser. No. 13/002,098, filed Mar. 7, 2011, which is a national stage application (under 35 U.S.C. 371) of PCT/EP2009/058534 filed Jul. 6, 2009, which claims benefit of German Application No. 102008031699.7 filed Jul. 4, 2008. The entire contents of each of these applications are hereby incorporated by reference herein in their entireties.

### SUBMISSION OF SEQUENCE LISTING

The Sequence Listing associated with this application is filed in electronic format via EFS-Web and hereby is incorporated by reference in its entirety into the specification. The name of the text file containing the Sequence Listing is 516154seqlist.txt. The size of the text file is 97.3 KB, and the text file was created on Mar. 30, 2015.

### FIELD OF THE INVENTION

The present invention relates to novel marker sequences for inflammatory prostate diseases, prostate carcinoma, and the diagnostic use thereof together with a method for screening potential active substances for prostate diseases of this type by means of these marker sequences. Furthermore, the invention relates to a diagnostic device containing marker sequences of this type for inflammatory prostate diseases and prostate carcinoma, in particular a protein biochip and the use thereof.

### BACKGROUND OF THE INVENTION

Protein biochips are gaining increasing industrial importance in analysis and diagnosis as well as in pharmaceutical development. Protein biochips have become established as screening instruments.

The rapid and highly parallel detection of a multiplicity of specifically binding analysis molecules in a single experiment is rendered possible hereby. To produce protein biochips, it is necessary to have the required proteins available. For this purpose, in particular protein expression libraries have become established. The high throughput cloning of defined open reading frames is one possibility (Heyman, J. A., Cornthwaite, J., Foncerrada, L., Gilmore, J. R., Gontang, E., Hartman, K. J., Hernandez, C. L., Hood, R., Hull, H. M., Lee, W. Y., Marcil, R., Marsh, E. J., Mudd, K. M., Patino, M. J., Purcell, T. J., Rowland, J. J., Sindici, M. L. and Hoeffler, J. P., (1999) Genome-scale cloning and expression of individual open reading frames using topoisomerase I-mediated ligation. Genome Res, 9, 383-392; Kersten, B., Feilner, T., Kramer, A., Wehrmeyer, S., Possling, A., Witt, I., Zanor, M. I., Stracke, R., Lueking, A., Kreutzberger, J., Lehrach, H. and Cahill, D. J. (2003) Generation of Arabidopsis protein chip for antibody and serum screening. Plant Molecular Biology, 52, 999-1010; Reboul, J., Reboul, J., Vaglio, P., Rual, J. F., Lamesch, P., Martinez, M., Armstrong, C M., Li, S., Jacotot, L., Bertin, N., Janky, R., Moore, T., Hudson, J. R., Jr., Hartley, J. L., Brasch, M. A., Vandenhaute, J., Boulton, S., Endress, G. A., Jenna, S., Chevet, E., Papasotiropoulos, V., Tolias, P. P., Ptacek, J., Snyder, M., Huang, R., Chance, M. R., Lee, H., Doucette-Stamm, L., Hill, D. E. and Vidal, M. (2003) C. elegans ORFeome Version 1.1: experimental verification of the genome annotation and resource for proteome-scale protein expression. Nat Genet, 34, 35-41.; Walhout, A. J., Temple, G. F., Brasch, M. A., Hartley, J. L., Lorson, M. A., van den Heuvel, S. and Vidal, M. (2000) GATEWAY recombinational cloning: application to the cloning of large numbers of open reading frames or ORFeomes. Methods Enzymol, 328, 575-592). However, an approach of this type is strongly connected to the progress of the genome sequencing projects and the annotation of these gene sequences. Furthermore, the determination of the expressed sequence can be ambiguous due to differential splicing processes. This problem may be circumvented by the application of cDNA expression libraries (Büssow, K., Cahill, D., Nietfeld, W., Bancroft, D., Scherzinger, E., Lehrach, H. and Walter, G. (1998) A method for global protein expression and antibody screening on high-density filters of an arrayed cDNA library. Nucleic Acids Research, 26, 5007-5008; Büssow, K., Nordhoff, E., Lübbert, C, Lehrach, H. and Walter, G. (2000) A human cDNA library for high-throughput protein expression screening. Genomics, 65, 1-8; Holz, C, Lueking, A., Bovekamp, L., Gutjahr, C, Bolotina, N., Lehrach, H. and Cahill, D. J. (2001) A human cDNA expression library in yeast enriched for open reading frames. Genome Res, 11, 1730-1735; Lueking, A., Holz, C, Gotthold, C, Lehrach, H. and Cahill, D. (2000) A system for dual protein expression in Pichia pastoris and Escherichia coli, Protein Expr. Purif., 20, 372-378). The cDNA of a particular tissue is hereby cloned into a bacterial or an eukaryotic expression vector, such as, e.g., yeast. The vectors used for the expression are generally characterized in that they carry inducible promoters that may be used to control the time of protein expression. Furthermore, expression vectors have sequences for so-called affinity epitopes or affinity proteins, which on the one hand permit the specific detection of the recombinant fusion proteins by means of an antibody directed against the affinity epitope, and on the other hand the specific purification via affinity chromatography (IMAC) is rendered possible.

For example, the gene products of a cDNA expression library from human fetal brain tissue in the bacterial expression system Escherichia coli were arranged in high-density format on a membrane and could be successfully screened with different antibodies. It was possible to show that the proportion of full-length proteins is at least 66%. Additionally, the recombinant proteins from the library could be expressed and purified in a high-throughput manner (Braun P., Hu, Y., Shen, B., Halleck, A., Koundinya, M., Harlow, E. and LaBaer, J. (2002) Proteome-scale purification of human proteins from bacteria. Proc Natl Acad Sci U S A, 99, 2654-2659; Büssow (2000) supra; Lueking, A., Horn, M., Eickhoff, H., Büssow, K., Lehrach, H. and Walter, G. (1999) Protein microarrays for gene expression and antibody screening. Analytical Biochemistry, 270, 103-111). Protein biochips of this type based on cDNA expression libraries are in particular the subject matter of WO 99/57311 and WO 99/57312.

Furthermore, in addition to antigen-presenting protein biochips, antibody-presenting arrangements are likewise described (Lal et al (2002) Antibody arrays: An embryonic but rapidly growing technology, DDT, 7, 143-149; Kusnezow et al. (2003), Antibody microarrays: An evaluation of production parameters, Proteomics, 3, 254-264).

However, there is a great need to provide indication-specific diagnostic devices, such as a protein biochip.

The laboratory parameters include acid phosphatase (AP) and prostate-specific antigen (PSA) for diagnosing prostate carcinoma. Above all, PSA currently has a high importance in diagnostics. It is specific for the prostate, but not for a tumor disease, but rather can also be elevated in the event of inflammation, benign prostate hyperplasia, urine retention, or without an obvious reason. A value over 4 ng/mL already requires clarification.

The object of the present invention is therefore to provide improved marker sequences and the diagnostic use thereof for the treatment of inflammatory prostate diseases up to prostate carcinoma.

The provision of specific marker sequences permits a reliable diagnosis and stratification of patients with inflammatory prostate diseases up to prostate carcinoma, in particular by means of a protein biochip.

### SUMMARY OF THE INVENTION

The invention therefore relates to the use of marker sequences for the diagnosis of inflammatory prostate diseases up to prostate carcinoma, wherein at least one marker sequence of a cDNA selected from the group SEQ ID NOS: 1-174 or respectively a protein coding therefor or respectively a partial sequence or fragment thereof (hereinafter: marker sequences according to the invention) is determined on or from a patient to be examined.

It was possible to identify the marker sequences according to the invention by means of differential screening of samples from healthy test subjects with patient samples with inflammatory prostate diseases, prostate carcinoma.

For the first time, these marker sequences according to the invention could be identified by means of protein biochips (see examples) hereby.

### BRIEF DESCRIPTION OF THE DRAWINGS

FIGS. 1A and 1B show the differential screening between two protein biochips from respectively one cDNA expression bank of a patient and a healthy test subject.

### DETAILED DESCRIPTION OF THE INVENTION

The term “inflammatory prostate diseases up to prostate carcinoma” comprises a group of diseases from prostatitis up to the chronic forms of all prostate inflammations and the establishment thereof as prostate cancer or prostate carcinoma (definition, e.g., according to Pschyrembel, de Gruyter, 261st edition (2007), Berlin).

In a further embodiment at least 2 to 5 or 10, preferably 30 to 50 marker sequences, or 50 to 100 or more marker sequences are determined on or from a patient to be examined.

In a further embodiment of the invention, the marker sequences according to the invention can likewise be combined, supplemented, fused, or expanded likewise with known biomarkers for this indication.

In a preferred embodiment, the determination of the marker sequences is carried out outside the human body and the determination is carried out in an ex vivo/in vitro diagnosis.

In a further embodiment of the invention, the invention relates to the use of marker sequences as diagnostic agents, wherein at least one marker sequence of a cDNA is selected from the group SEQ ID NOS: 1-174 or respectively a protein coding therefor or respectively a partial sequence or fragment thereof.

Furthermore, the invention relates to a method for the diagnosis of inflammatory prostate diseases up to prostate carcinoma, wherein a.) at least one marker sequence of a cDNA selected from the group SEQ ID NOS: 1-174 or respectively a protein coding therefor or respectively a partial sequence or fragment thereof is applied to a solid support and b.) is brought into contact with body fluid or tissue extract of a patient and c.) the detection of an interaction of the body fluid or tissue extract with the marker sequences from a.) is carried out.

The invention therefore likewise relates to diagnostic agents for the diagnosis of inflammatory prostate diseases up to prostate carcinoma respectively selected from the group SEQ ID NOS: 1-174 or respectively a protein coding therefor or respectively a partial sequence or fragment thereof.

In a particularly preferred embodiment, the marker sequences SEQ ID NOS: 136, 40, 127, 83, 16, 82, 88, 152, 130, 138, 2, 12, 113, 20, 173, 33, 172, 52, 43, 91, 1, 32, 86, 27, 105 are preferred in this order.

The detection of an interaction of this type can be carried out, for example, by a probe, in particular by an antibody.

The invention therefore likewise relates to the object of providing a diagnostic device or an assay, in particular a protein biochip, which permits a diagnosis or examination for inflammatory prostate diseases up to prostate carcinoma.

Furthermore, the invention relates to a method for the stratification, in particular risk stratification and/or therapy control of a patient with inflammatory prostate diseases up to prostate carcinoma, wherein at least one marker sequence of a cDNA selected from the group SEQ ID NOS: 1-174 or respectively a protein coding therefor is determined on a patient to be examined.

Furthermore, the stratification of the patients with inflammatory prostate diseases up to prostate carcinoma in new or established subgroups of inflammatory prostate diseases up to prostate carcinoma is also covered, as well as the expedient selection of patient groups for the clinical development of novel therapeutic agents. The term therapy control likewise covers the allocation of patients to responders and non-responders regarding a therapy or the therapy course thereof.

“Diagnosis” for the purposes of this invention means the positive determination of inflammatory prostate diseases up to prostate carcinoma by means of the marker sequences according to the invention as well as the assignment of the patients to inflammatory prostate diseases up to prostate carcinoma. The term diagnosis covers medical diagnostics and examinations in this regard, in particular in-vitro diagnostics and laboratory diagnostics, likewise proteomics and nucleic acid blotting. Further tests can be necessary to be sure and to exclude other diseases. The term diagnosis therefore likewise covers the differential diagnosis of inflammatory prostate diseases, prostate carcinoma by means of the marker sequences according to the invention and the prognosis of inflammatory prostate diseases and prostate carcinoma.

“Stratification or therapy control” for the purposes of this invention means that the method according to the invention renders possible decisions for the treatment and therapy of the patient, whether it is the hospitalization of the patient, the use, effect and/or dosage of one or more drugs, a therapeutic measure or the monitoring of a course of the disease and the course of therapy or etiology or classification of a disease, e.g., into a new or existing subtype or the differentiation of diseases and the patients thereof.

In a further embodiment of the invention, the term “stratification” covers in particular the risk stratification with the prognosis of an outcome of a negative health event.

Within the scope of this invention, “patient” means any test subject—human or mammal—with the proviso that the test subject is tested for inflammatory prostate diseases up to prostate carcinoma.

The term “marker sequences” for the purposes of this invention means that the cDNA or the polypeptide or protein that can be respectively obtained therefrom are significant for inflammatory prostate diseases, prostate carcinoma. For example, the cDNA or the polypeptide or protein that can be respectively obtained therefrom can exhibit an interaction with substances from the body fluid or tissue extract of a patient with inflammatory prostate diseases, prostate carcinoma (e.g., antigen (epitope)/antibody (paratope) interaction). For the purposes of the invention “wherein at least one marker sequence of a cDNA selected from the group SEQ ID NOS: 1-174 or respectively a protein coding therefor or respectively a partial sequence or fragment thereof is determined on a patient to be examined” means that an interaction between the body fluid or tissue extract of a patient and the marker sequences according to the invention is detected. An interaction of this type is, e.g., a bond, in particular a binding substance on at least one marker sequence according to the invention or in the case of a cDNA the hybridization with a suitable substance under selected conditions, in particular stringent conditions (e.g., such as usually defined in J. Sambrook, E. F. Fritsch, T. Maniatis (1989), Molecular cloning: A laboratory manual, 2nd Edition, Cold Spring Harbor Laboratory Press, Cold Spring Harbor, USA or Ausubel, “Current Protocols in Molecular Biology,” Green Publishing Associates and Wiley Interscience, N.Y. (1989)). One example of stringent hybridization conditions is: hybridization in 4×SSC at 65° C. (alternatively in 50% formamide and 4×SSC at 42° C.), followed by several washing steps in 0.1×SSC at 65° C. for a total of approximately one hour. An example of less stringent hybridization conditions is hybridization in 4×SSC at 37° C., followed by several washing steps in 1×SSC at room temperature.

According to the invention, substances of this type are constituents of a body fluid, in particular blood, whole blood, blood plasma, blood serum, patient serum, urine, cerebrospinal fluid, synovial fluid, or of a tissue extract of the patient.

In a further embodiment of the invention, however, the marker sequences according to the invention can be present in a significantly higher or lower expression rate or concentration that indicates inflammatory prostate diseases, prostate carcinoma. The relative sick/healthy expression rates of the marker sequences for inflammatory prostate diseases, prostate carcinoma according to the invention are hereby determined by means of proteomics or nucleic acid blotting.

In a further embodiment of the invention, the marker sequences have a recognition signal that is addressed to the substance to be bound (e.g., antibody, nucleic acid). It is preferred according to the invention that for a protein the recognition signal is an epitope and/or a paratope and/or a hapten and for a cDNA is a hybridization or binding region.

The marker sequences according to the invention are the subject matter of Table A and can be clearly identified by the respectively cited database entry (also by means of the Internet: ncbi.nlm.nih.gov) (see in Table A: accession no. there), see also the associated sequence protocol.

The invention therefore also relates to the full-length sequences of the markers according to the invention, as defined in Table 1 via the known database entry according to Table A.

Therefore, the invention also comprises analogous embodiments of the sequences of the known database entries listed in Table A to the marker sequences SEQ ID NOS: 1-174, such as, e.g., described in the claims, since the SEQ ID NOS: 1-174 according to the invention in turn represent partial sequences, at least with high homology. The specific marker sequences SEQ ID NOS: 1-174 are preferred according to the invention, however.

Furthermore, the following sequences of the known database entries listed in Table A are preferred: gi:61102726, gi:23308566, gi:56117827 (SEQ ID NO: 175), gi:40789263, gi:13375724, gi:40354199, gi:41393564, gi:7705400, gi:57617038, gi:63082031, gi:113413768, gi:12751496, gi:50878292, gi:14251213, gi:94536841, gi:22035555, gi:90903237, gi:30089990, gi:24308256, gi:44917603, gi:13402448, gi:22027484, gi:41352062, gi:19743568, and gi:47717133.

According to the invention, the marker sequences also cover those modifications of the cDNA sequence and the corresponding amino acid sequence as chemical modification, such as citrullination, acetylation, phosphorylation, glycosylation or poly(A) strand and other modifications known to one skilled in the art.

In a further embodiment of the invention, partial sequences or fragments of the marker sequences according to the invention are likewise comprised. In particular those partial sequences that have an identity of 95%, 90%, in particular 80% or 70% with the marker sequences according to the invention.

Partial sequences are also sequences of the type which have 50 to 100 nucleotides, 70-120 nucleotides of a sequence of the SEQ ID NOS: 1-174, or peptides obtainable therefrom.

In a further embodiment, the respective marker sequence can be represented in different quantities in one more regions on a solid support. This permits a variation of the sensitivity. The regions can have respectively a totality of marker sequences, i.e., a sufficient number of different marker sequences, in particular 2 to 5 or 10 or more and optionally more nucleic acids and/or proteins, in particular biomarkers. However, at least 96 to 25,000 (numerical) or more from different or identical marker sequences and further nucleic acids and/or proteins, in particular biomarkers are preferred. Furthermore preferred are more than 2,500, in particular preferred 10,000 or more different or identical marker sequences and optionally further nucleic acids and/or proteins, in particular biomarkers.

Another object of the invention relates to an arrangement of marker sequences containing at least one marker sequence of a cDNA selected from the group SEQ ID NOS: 1-174 or respectively a protein coding therefor. Preferably, the arrangement contains at least 2 to 5 or 10, preferably 30 to 50 marker sequences, or 50 to 100 or more marker sequences.

Within the scope of this invention, “arrangement” is synonymous with “array,” and if this “array” is used to identify substances on marker sequences, this is to be understood to be an “assay” or diagnostic device. In a preferred embodiment, the arrangement is designed such that the marker sequences represented on the arrangement are present in the of a grid on a solid support. Furthermore, those arrangements are preferred that permit a high-density arrangement of protein binders and the marker sequences are spotted. Such high-density spotted arrangements are disclosed, for example, in WO 99/57311 and WO 99/57312 and can be used advantageously in a robot-supported automated high-throughput method.

Within the scope of this invention, however, the term “assay” or diagnostic device likewise comprises those embodiments of a device, such as ELISA, bead-based assay, line assay, Western Blot, immunochromatographic methods (e.g., so-called lateral flow immunoassays, or similar immunological single or multiplex detection measures. A protein biochip in terms of this invention is the systematic arrangement of proteins on a solid support.

The marker sequences of the arrangement are fixed on a solid support, but preferably spotted or immobilized even printed on, i.e. applied in a reproducible manner. One or more marker sequences can be present multiple times in the totality of all marker sequences and present in different quantities based on one spot. Furthermore, the marker sequences can be standardized on the solid support (i.e., by means of serial dilution series of, e.g., human globulins as internal calibrators for data normalization and quantitative evaluation).

The invention therefore relates to an assay or a protein biochip comprising an arrangement containing marker sequences according to the invention.

In a further embodiment, the marker sequences are present as clones. Clones of this type can be obtained, for example, by means of a cDNA expression library according to the invention (Büssow et al. 1998 (supra)). In a preferred embodiment, such expression libraries containing clones are obtained using expression vectors from a cDNA expression library comprising the cDNA marker sequences. These expression vectors preferably contain inducible promoters. The induction of the expression can be carried out, e.g., by means of an inductor, such as IPTG. Suitable expression vectors are described in Terpe et al. (Terpe T Appl Microbiol Biotechnol. 2003 January; 60(5): 523-33).

One skilled in the art is familiar with expression libraries, they can be produced according to standard works, such as Sambrook et al, “Molecular Cloning, A laboratory handbook, 2nd edition (1989), CSH press, Cold Spring Harbor, N.Y. Expression libraries are also preferred which are tissue-specific (e.g., human tissue, in particular human organs). Furthermore included according to the invention are expression libraries that can be obtained by exon-trapping. A synonym for expression library is expression bank. Also preferred are protein biochips or corresponding expression libraries that do not exhibit any redundancy (so-called: UNICLONE® library) and that may be produced, for example, according to the teachings of WO 99/57311 and WO 99/57312. These preferred UNICLONE libraries have a high portion of non-defective fully expressed proteins of a cDNA expression library.

Within the context of this invention, the clones can also be, but not limited to, transformed bacteria, recombinant phages, or transformed cells from mammals, insects, fungi, yeasts, or plants.

The clones are fixed, spotted, or immobilized on a solid support.

The invention therefore relates to an arrangement wherein the marker sequences are present as clones.

Additionally, the marker sequences can be present in the respective form of a fusion protein, which contains, for example, at least one affinity epitope or tag. The tag may be one such as contains c-myc, his tag, arg tag, FLAG, alkaline phosphatase, VS tag, T7 tag or strep tag, HAT tag, NusA, S tag, SBP tag, thioredoxin, DsbA, a fusion protein, preferably a cellulose-binding domain, green fluorescent protein, maltose-binding protein, calmodulin-binding protein, glutathione S-transferase, or lacZ.

In all of the embodiments, the term “solid support” covers embodiments such as a filter, a membrane, a magnetic or fluorophore-labeled bead, a silica wafer, glass, metal, ceramics, plastics, a chip, a target for mass spectrometry, or a matrix. However, a filter is preferred according to the invention.

As a filter, furthermore PVDF, nitrocellulose, or nylon is preferred (e.g., Immobilon P Millipore, Protran Whatman, Hybond N+ Amersham).

In another preferred embodiment of the arrangement according to the invention, the arrangement corresponds to a grid with the dimensions of a microtiter plate (8-12 wells strips, 96 wells, 384 wells, or more), a silica wafer, a chip, a target for mass spectrometry, or a matrix.

In a further embodiment, the invention relates to an assay or a protein biochip for identifying and characterizing a substance for inflammatory prostate diseases, prostate carcinoma, characterized in that an arrangement or assay according to the invention is a.) brought into contact with at least one substance to be tested and b.) a binding success is detected.

Furthermore, the invention relates to a method for identifying and characterizing a substance for inflammatory prostate diseases, prostate carcinoma, characterized in that an arrangement or assay according to the invention is a.) brought into contact with at least one substance to be tested and b.) a binding success is detected.

The substance to be tested can be any native or non-native biomolecule, a synthetic chemical molecule, a mixture, or a substance library.

After the substance to be tested contacts a marker sequence, the binding success is evaluated, which, for example, is carried out using commercially available image analyzing software (GenePix Pro (Axon Laboratories), Aida (Ray test), ScanArray (Packard Bioscience)).

The visualization of protein-protein interactions according to the invention (e.g., protein on marker sequence, as antigen/antibody) or corresponding “means for detecting the binding success” can be performed, for example, using fluorescence labeling, biotinylation, radioisotope labeling, or colloid gold or latex particle labeling in the usual way. A detection of bound antibodies is carried out with the aid of secondary antibodies, which are labeled with commercially available reporter molecules (e.g., Cy, Alexa, Dyomics, FITC, or similar fluorescent dyes, colloidal gold or latex particles), or with reporter enzymes, such as alkaline phosphatase, horseradish peroxidase, etc., and the corresponding colorimetric, fluorescent, or chemiluminescent substrates. Readout is conducted, e.g., using a microarray laser scanner, a CCD camera, or visually.

In a further embodiment, the invention relates to a drug/active substance or prodrug developed for inflammatory prostate diseases, prostate carcinoma and obtainable through the use of the assay or protein biochip according to the invention.

The invention therefore likewise relates to the use of an arrangement according to the invention or an assay for screening active substances for inflammatory prostate diseases, prostate carcinoma.

In a further embodiment, the invention therefore likewise relates to a target for the treatment and therapy of inflammatory prostate diseases, prostate carcinoma respectively selected from the group SEQ ID NOS: 1-174 or a protein respectively coding therefor.

In a further embodiment, the invention likewise relates to the use of the marker sequences according to the invention, preferably in the form of an arrangement, as an affinity material for carrying out an apheresis or in the broadest sense a blood lavage, wherein substances from body fluids of a patient with inflammatory prostate diseases, prostate carcinoma, such as blood or plasma, bind to the marker sequences according to the invention and consequently can be selectively withdrawn from the body fluid.

### Examples and Figures

Ten or more patient samples were individually screened against a cDNA expression library. The expression clones specific to inflammatory prostate diseases, prostate carcinoma were determined through a comparison with ten or more healthy samples. The identity of the marker sequences was determined by DNA sequencing.

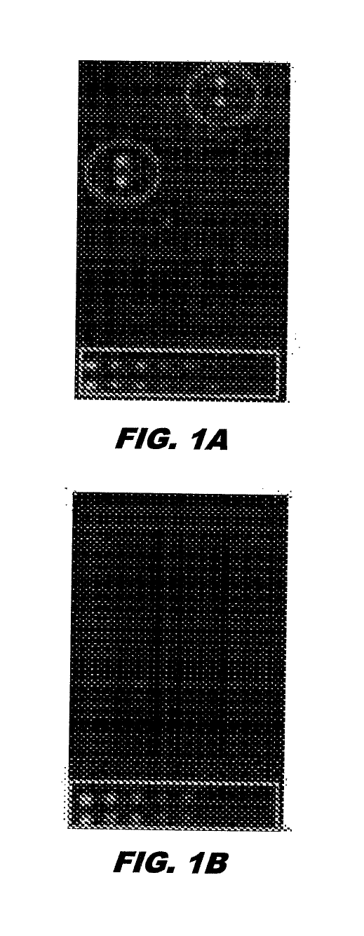

FIGS. 1A and 1B show the differential screening between two protein biochips from respectively one cDNA expression bank of a patient and a healthy test subject. The differential clones are detected by means of fluorescent labeling and evaluated by means of bioinformatics.

In the scope of the biomarker identification, various bioinformatic analyses are performed. For each serum, reactivities against approximately 2000 different antigens are measured by means of microarray. These data are used for a ranking of the spotted antigens with respect to their differentiation capability between healthy and diseased sera. This analysis is performed by means of the non-parameterized Mann-Whitney test on normalized intensity data. An internal standard which is also spotted on each chip is used for the normalization. Since a p value is calculated for each antigen, methods are used for correction of the multiple test. As a very conservative approach, a Bonferroni direction is performed and the less restrictive false discovery rate (FDR) according to Benjamini & Hochberg is additionally calculated. Furthermore, the data are used for classification of the sera. Different multivariate methods are used hereby. These are methods from statistical learning methods such as support vector machines (SVM), neural networks, or classification trees, as well as a threshold value method, which is capable of both classification and also visual representation of the data.

To avoid overfitting, a 10-fold cross-validation of the data is performed.

TABLE A

| SEQ | | | |
|---|---|---|---|
| ID | | | |
| NO: | Accession | Blast | Clone |
| N/A | gi\|13402448 | PREDICTED: Homo sapiens similar to CXYorf1-related | 00800 578 N18 |
| | | protein (LOC653635), mRNA | |
| N/A | gi\|113413768 | PREDICTED: Homo sapiens family with sequence | 00800 570 014 |
| | | similarity 59, member B (FAM59B), mRNA | |
| N/A | gi\|113414262 | PREDICTED: Homo sapiens SPEG complex locus | 00800 557 N13 |
| | | (SPEG), mRNA | |
| N/A | gi\|113418314 | PREDICTED: Homo sapiens NHS-like 1, transcript | 00800 507 H03 |
| | | variant 5 (NHSL1), mRNA | |
| N/A | gi\|113425012 | PREDICTED: Homo sapiens kinesin family member 26A | 00800 583 H21 |
| | | (KIF26A), mRNA | |
| N/A | gi\|13426606 | PREDICTED: Homo sapiens hypothetical protein | 00800 550 A18 |
| | | LOC727910 (LOC727910), mRNA | |
| N/A | gi\|113427260 | PREDICTED: Homo sapiens jumonji domain containing | 00800 569 A13 |
| | | 3, transcript variant 3 (JMJD3), mRNA | |
| N/A | gi\|113428505 | PREDICTED: Homo sapiens widely-interspaced zinc | 00800 588 F10 |
| | | finger motifs, transcript variant 10 (WIZ), mRNA | |
| N/A | gi\|113428756 | PREDICTED: Homo sapiens zinc finger protein 154 | 00800 597 K23 |
| | | (pHZ-92) (ZNF154), mRNA | |
| N/A | gi\|l13429538 | PREDICTED: Homo sapiens tetratricopeptide repeat | 00800 556 D03 |
| | | domain 28 (TTC28), mRNA | |
| 11 | gi\|113431093 | PREDICTED: Homo sapiens GIY-YIG domain | 00800 514 H03 |
| | | containing 2, transcript variant 1 (GIYD2), mRNA | |
| N/A | gi\|12751496 | Homo sapiens chromosome 8 open reading frame 33 | 00800 601 K04 |
| | | (C8orf33), mRNA | |
| N/A | gi\|13325056 | Homo sapiens solute carrier family 27 (fatty acid | 00800 520 A13 |
| | | transporter), member 5 (SLC27A5), mRNA | |
| N/A | gi\|13325058 | Homo sapiens ARP1 actin-related protein 1 homolog A, | 00800 582 L08 |
| | | centractin alpha (yeast) (ACTR1A), mRNA | |
| N/A | gi\|13375663 | Homo sapiens family with sequence similarity 77, | 00800 586 F01 |
| | | member C (FAM77C), mRNA | |
| N/A | gi\|13375724 | Homo sapiens chromosome 14 open reading frame 138 | 00800 528 C03 |
| | | (C14orf138), mRNA | |
| N/A | gi\|13904863 | Homo sapiens cytochrome P450, family 27, subfamily A, | 00800 525 H24 |
| | | polypeptide 1 (CYP27A1), nuclear gene encoding | |
| | | mitochondrial protein, mRNA | |
| N/A | gi\|14042967 | Homo sapiens spinster (SPIN1), mRNA | 00800 531 P04 |
| N/A | gi\|14110410 | Homo sapiens heterogeneous nuclear ribonucleoprotein | 00800 520 A20 |
| | | D-like (HNRPDL), transcript variant 1, mRNA | |
| N/A | gi\|14251213 | Homo sapiens DEAD (Asp-Glu-Ala-Asp) box | 00800 592 F13 |
| | | polypeptide 24 (DDX24), mRNA | |
| N/A | gi\|14591916 | Homo sapiens ribosomal protein S25 (RPS25), mRNA | 00800 553 C23 |
| N/A | gi\| 4772189 | Homo sapiens chromosome 20 genomic contig, | 00800 530 J21 |
| | | reference assembly | |
| N/A | gi\|15431299 | Homo sapiens ribosomal protein L 18a (RPL 18A), | 00800 564 D11 |
| | | mRNA | |
| N/A | gi\|16905511 | Homo sapiens ribosomal protein, large, P1 (RPLP1), | 00800 530 C03 |
| | | transcript variant 1, mRNA | |
| N/A | gi\|17149837 | Homo sapiens FK506 binding protein 1 A, 12kDa | 00800 598 J17 |
| | | (FKBP1A), transcript variant 12B, mRNA | |
| N/A | gi\|18390348 | Homo sapiens ribosomal protein L7a (RPL7A), mRNA | 00800 528 A14 |
| N/A | gi\| 19743568 | Homo sapiens TRAF family member-associated NFKB | 00800 541 P08 |
| | | activator (TANK), transcript variant 1, m RNA | |
| N/A | gi\|20357526 | Homo sapiens guanine nucleotide binding protein (G | 00800 583 H15 |
| | | protein), beta polypeptide 1 (GNB1), mRNA | |
| N/A | gi\|21 071 045 | Homo sapiens SWI/SNF related, matrix associated, | 00800 588 J12 |
| | | actin dependent regulator of chromatin, subfamily a, | |
| | | member 1 (SMARCA1), transcript variant 2, mRNA | |
| N/A | gi\|21361156 | Homo sapiens homer homolog 3 (Drosophila) | 00800 518 010 |
| | | (HOMER3), mRNA | |
| N/A | gi\|21389314 | Homo sapiens solute carrier family 25 (mitochondrial | 00800 583 B14 |
| | | carrier; citrate transporter), member 1 (SLC25A 11, | |
| | | mRNA | |
| N/A | gi\|22027484 | Homo sapiens RAS, dexamethasone-induced 1 | 00800 564 E02 |
| | | (RASD1), mRNA | |
| N/A | gi\|22035555 | Homo sapiens BRF1 homolog, subunit of RNA | 00800 525 C01 |
| | | polymerase III transcription initiation factor IIIB (S. | |
| | | cerevisiae) (BRF1), transcript variant 1, mRNA | |
| N/A | gi\|22095372 | Homo sapiens LIM domain containing 2 (LIMD2), mRNA | 00800 568 D15 |
| N/A | gi\|22202623 | Homo sapiens glutathione transferase zeta 1 | 00800 547 A15 |
| | | (maleylacetoacetate isomerase) (GSTZ1), transcript | |
| | | variant 1, mRNA | |
| N/A | gi\|22212935 | Homo sapiens opioid receptor, sigma 1 (OPRS1), | 00800 523 E04 |
| | | transcript variant 3, mRNA | |
| N/A | gi\|22538452 | Homo sapiens phosphatidylinositol glycan anchor | 00800 574 K18 |
| | | biosynthesis, class 0 (PIGO), transcript variant 1, mRNA | |
| N/A | gi\|23111017 | Homo sapiens RNA binding motif protein 10 (RBM1 0), | 00800 586 M11 |
| | | transcript variant 2, mRNA | |
| N/A | gi\|23238227 | Homo sapiens carbohydrate (N-acetylglucosamine 6-0) | 00800 540 C04 |
| | | sulfotransferase 7 (CHST7), mRNA | |
| N/A | gi\|23308566 | Homo sapiens asparaQinase like 1 (ASRGL 1), mRNA | 00800 563 013 |
| N/A | gi\|24234719 | Homo sapiens DnaJ (Hsp40) homolog, subfamily B, | 00800 528 M21 |
| | | member 6 (DNAJB6), transcript variant 2, mRNA | |
| N/A | gi\|24308032 | Homo sapiens formin binding protein 4 (FNBP4), mRNA | 00800 520 D08 |
| N/A | gi\|24308256 | Homo sapiens KIAA1576 protein (KIAA1576), mRNA | 00800 590 P16 |
| N/A | gi\|24475884 | Homo sapiens Ras association (RaIGDS/AF-6) domain | 00800 512 C23 |
| | | family 7 (RASSF7), mRNA | |
| N/A | gi\|27886683 | Homo sapiens Kv channel interacting protein 1 | 00800 585 M21 |
| | | (KCNIP1), transcript variant 2, m RNA | |
| N/A | gi\|128178831 | Homo sapiens isocitrate dehydrogenase 2 (NADP+), | 00800 532 L07 |
| | | mitochondrial (IDH2), mRNA | |
| N/A | gi\|28269671 | Homo sapiens serologically defined colon cancer | 00800 589 C17 |
| | | antigen 8 (SDCCAG8), mRNA | |
| N/A | gi\|28872795 | Homo sapiens CCAAT/enhancer binding protein | 00800 599 K11 |
| | | (C/EBP), beta (CEBPB), mRNA | |
| N/A | gi\|29800963 | Homo sapiens chromosome 10 genomic contig, | 00800 584 124 |
| | | reference assembly | |
| N/A | gi\|29826322 | Homo sapiens adducin 1 (alpha) (ADD1), transcript | 00800 602 C16 |
| | | variant 3, mRNA | |
| N/A | gi\|29826324 | Homo sapiens adducin 1 (alpha) (ADD1), transcript | 00800 506 H06 |
| | | variant 4, mRNA | |
| N/A | gi\|30089990 | Homo sapiens acid phosphatase 1, soluble (ACP1), | 00800 601 N08 |
| | | transcript variant 3, mRNA | |
| N/A | gi\|30795226 | Homo sapiens histidyl-tRNA synthetase 2 (HARS2), | 00800 578 C22 |
| | | mRNA | |
| N/A | gi\|31083149 | Homo sapiens axin 1 (AXIN1), transcript variant 1, | 00800 555 A23 |
| | | mRNA | |
| N/A | gi\|31341380 | Homo sapiens sterile alpha motif domain containing 14 | 00800 530 E15 |
| | | (SAMD14), mRNA | |
| N/A | gi\|31377576 | Homo sapiens chromosome 10 open reading frame 13 | 00800 533 B02 |
| | | (C1 Oorf13), m RNA | |
| N/A | gi\|31543618 | Homo sapiens splicing factor, arginine/serine-rich 1 | 00800 512 M22 |
| | | (splicing factor 2, alternate splicing factor) (SFRS1), | |
| | | mRNA | |
| N/A | gi\|31982913 | Homo sapiens WD repeat domain 54 (WDR54), mRNA | 00800 590 J15 |
| N/A | gi\|32171243 | Homo sapiens hypothetical protein DKFZp434G156 | 00800 537 L05 |
| | | (NAG6), mRNA | |
| N/A | gi\|32261293 | Homo sapiens protein kinase, interferon-inducible | 00800 522 G17 |
| | | double stranded RNA dependent activator (PRKRA), | |
| | | mRNA | |
| N/A | gi\|32454740 | Homo sapiens serpin peptidase inhibitor, Glade H (heat | 00800 524 110 |
| | | shock protein 47), member 1, (collagen binding protein | |
| | | 1) (SERPINH1), mRNA | |
| N/A | gi\|32490571 | Homo sapiens erythrocyte membrane protein band 4.1- | 00800 567 M08 |
| | | like 3 (EPB41 L3), mRNA | |
| N/A | gi\|33469963 | Homo sapiens splicing factor 4 (SF4), mRNA | 00800 518 L01 |
| N/A | gi\|33469975 | Homo sapiens activating transcription factor 4 (tax- | 00800 570 D17 |
| | | responsive enhancer element B67) (ATF4), transcript | |
| | | variant 1, mRNA | |
| N/A | gi\|33469983 | Homo sapiens protein disulfide isomerase family A, | 00800 600 D18 |
| | | member 4 (PDIA4), mRNA | |
| N/A | gi\|33598947 | Homo sapiens phospholipase C, gamma 1 (PLCG1), | 00800 536 F02 |
| | | transcript variant 1, m RNA | |
| N/A | gi\|34147350 | Homo sapiens RAS-like, family 11, member B (RASL11 | 00800 601 M15 |
| | | B), mRNA | |
| N/A | gi\|34147700 | Homo sapiens dehydrogenase/reductase (SDR family) | 00800 578 D10 |
| | | member 13 (DHRS13), mRNA | |
| N/A | gi\|34222379 | Homo sapiens family with sequence similarity 100, | 00800 594 J07 |
| | | member B (FAM1 00B), mRNA | |
| N/A | gi\|34452731 | Homo sapiens phosphatidylinositol 3,4,5-trisphosphate- | 00800 530 E24 |
| | | dependent RAC exchanger 1 (PREX1), mRNA | |
| N/A | gi\|37550981 | Homo sapiens chromosome 10 genomic contig, | 00800 596 A22 |
| | | reference assembly | |
| N/A | gi\|37551026 | Homo sapiens chromosome 10 genomic contig, | 00800 540 H15 |
| | | reference assembly | |
| N/A | gi\|38372936 | Homo sapiens chromatin modifying protein 2A | 00800 533 P22 |
| | | (CHMP2A), transcript variant 1, m RNA | |
| N/A | gi\|38372939 | Homo sapiens alpha-2-glycoprotein 1, zinc (AZGP1), | 00800 519 J13 |
| | | mRNA | |
| N/A | gi\|38524584 | Homo sapiens NAOH dehydrogenase (ubiquinone) Fe-S | 00800 541 B07 |
| | | protein 7, 20kOa (NAOH-coenzyme Q reductase) | |
| | | (NOUFS7), mRNA | |
| N/A | gi\|38569414 | Homo sapiens amyloid beta (A4) precursor protein- | 00800 582 B04 |
| | | binding, family A, member 2 binding protein (APBA2BP), | |
| | | transcript variant 2, mRNA | |
| N/A | gi\|38679885 | Homo sapiens splNryanodine receptor domain and | 00800 509 010 |
| | | SOCS box containing 3 (SPSB3), mRNA | |
| N/A | gi\|38679891 | Homo sapiens protein (peptidylprolyl cis/trans | 00800 579 A06 |
| | | isomerase) NIMA-interacting, 4 (parvulin) (PIN4), mRNA | |
| N/A | gi\|38679903 | Homo sapiens AOP-ribosylation factor-like 8A (ARL8A), | 00800 562 N23 |
| | | mRNA | |
| N/A | gi\|38683848 | Homo sapiens fibroblast growth factor (acidic) | 00800 527 J24 |
| | | intracellular binding protein (FIBP), transcript variant 1, | |
| | | mRNA | |
| N/A | gi\|38788107 | Homo sapiens small glutamine-rich tetratricopeptide | 00800 577 P08 |
| | | repeat (TPR)-containing, alpha (SGTA), mRNA | |
| N/A | gi\|40354199 | Homo sapiens TPX2, microtubule-associated, homolog | 00800 541 F11 |
| | | (Xenopus laevis) (TPX2), mRNA | |
| N/A | gi\|40789263 | Homo sapiens hypothetical protein MGC11257 | 00800 511 M24 |
| | | (MGC11257), mRNA | |
| N/A | gi\|40795666 | Homo sapiens ubiquitin specific peptidase 4 (proto- | 00800 562 E18 |
| | | oncogene) (USP4), transcript variant 2, mRNA | |
| N/A | gi\|40805842 | Homo sapiens p300/CBP-associated factor (PCAF), | 00800 578 M10 |
| | | mRNA | |
| N/A | gi\|41352062 | Homo sapiens phosphofructokinase, platelet (PFKP), | 0800 548 E23 |
| | | mRNA | |
| N/A | gi\|41352714 | Homo sapiens vacuolar protein sorting 35 (yeast) | 00800 586 A05 |
| | | (VPS35), mRNA | |
| N/A | gi\|41393564 | Homo sapiens inositol 1 ,3,4-triphosphate 5/6 kinase | 00800 578 K17 |
| | | (ITPK1), mRNA | |
| N/A | gi\|41406095 | Homo sapiens OEAH (Asp-Glu-Ala-His) box polypeptide | 00800 586 C18 |
| | | 38 (OHX38), mRNA | |
| N/A | gi\|42734426 | Homo sapiens NGFI-A binding protein 2 (EGR1 binding | 00800 570 C19 |
| | | protein 2) (NAB2), mRNA | |
| N/A | gi\|44917603 | Homo sapiens SLiT-ROBO Rho GTPase activating | 00800 574 117 |
| | | protein 1 (SRGAP1), mRNA | |
| N/A | gi\|4504618 | Homo sapiens insulin-like growth factor binding protein | 00800 524 E19 |
| | | 7 (IGFBP7), mRNA | |
| N/A | gi\|4505324 | Homo sapiens Sjogren's syndrome nuclear autoantigen | 00800 541 N09 |
| | | 1 (SSNA1), mRNA | |
| N/A | gi\|4507126 | Homo sapiens small nuclear ribonucleoprotein | 00800 529 022 |
| | | polypeptide C (SNRPC), mRNA | |
| N/A | gi\|45439358 | Homo sapiens triple functional domain (PTPRF | 00800 546 115 |
| | | interacting) (TRIO), mRNA | |
| N/A | gi\|4557766 | Homo sapiens methylmalonyl Coenzyme A mutase | 00800 520 001 |
| | | (MUT), nuclear gene encoding mitochondrial protein, | |
| | | mRNA | |
| N/A | gi\|4557788 | Homo sapiens Norrie disease (pseudoglioma) (NOP), | 00800 598 K21 |
| | | mRNA | |
| N/A | gi\|45597176 | Homo sapiens TBC1 domain family, member 9B (with | 00800 549 J10 |
| | | GRAM domain) (TBC1 09B), transcript variant 2, mRNA | |
| N/A | gi\|46198303 | Homo sapiens coiled-coil-helix-coiled-coil-helix domain | 00800 601 M20 |
| | | containing 8 (CHCHD8), mRNA | |
| N/A | gi\|46370090 | Homo sapiens chromosome 11 open reading frame 31 | 00800 586 C20 |
| | | (C11orf31), mRNA | |
| N/A | gi\|46411160 | Homo sapiens aconitase 2, mitochondrial (AC02), | 00800 578 019 |
| | | nuclear gene encoding mitochondrial protein, mRNA | |
| N/A | gi\|47132573 | Homo sapiens protein kinase, AMP-activated, gamma 1 | 00800 583 109 |
| | | non-catalytic subunit (PRKAG1), transcript variant 1, | |
| | | mRNA | |
| N/A | gi\|47132588 | Homo sapiens protein kinase N1 (PKN1), transcript | 00800 585 K02 |
| | | variant 2, mRNA | |
| N/A | gi\|4757793 | Homo sapiens acetylserotonin O-methyltransferase-like | 00800 566 K12 |
| | | (ASMTL), mRNA | |
| N/A | gi\|47717133 | Homo sapiens CDC-like kinase 2 (CLK2), transcript | 00800 539 C01 |
| | | variant 1, mRNA | |
| | | mRNA | |
| N/A | gi\|47933338 | Homo sapiens RNA binding motif protein 15 (RBM15), | 00800 587 F09 |
| N/A | gi\|48527950 | Homo sapiens golgi associated, gamma adaptin ear | 00800 595 F17 |
| | | containing, ARF binding protein 1 (GGA 1), transcript | |
| | | variant 1, mRNA | |
| N/A | gi\|48675816 | Homo sapiens hypothetical protein FLJ1 0154 (FLJ1 | 00800 552 M12 |
| | | 0154), mRNA | |
| N/A | gi\|49355764 | Homo sapiens ELAV (embryonic lethal, abnormal vision, | 00800 506 024 |
| | | Drosophila)-like 3 (Hu antigen C) (ELAVL3), transcript | |
| | | variant 2, mRNA | |
| N/A | gi\|50053889 | Homo sapiens chromosome 14 open reading frame 131 | 00800 545 A12 |
| | | (C14orf131), mRNA | |
| N/A | gi\|5032030 | Homo sapiens RNA binding motif protein 5 (RBM5), | 00800 506 B06 |
| | | mRNA | |
| N/A | gi\|50345295 | Homo sapiens complement component 4B (Childo blood | 00800 602 A21 |
| | | group) (C4B), mRNA | |
| N/A | gi\|50878292 | Homo sapiens tripartite motif-containing 45 (TRIM45), | 00800 529 L23 |
| | | mRNA | |
| N/A | gi\|51464897 | Homo sapiens chromosome 5 genomic contig, | 00800 516 H09 |
| | | reference assembly | |
| N/A | gi\|51466739 | Homo sapiens chromosome 8 genomic contig, | 00800 573 P03 |
| | | reference assembly | |
| N/A | gi\|51467074 | Homo sapiens chromosome 8 genomic contig, | 00800 579 H02 |
| | | reference assembly | |
| N/A | gi\|51473102 | Homo sapiens chromosome 16 genomic contig, | 00800 583 L05 |
| | | reference assembly | |
| N/A | gi\|51473128 | Homo sapiens chromosome 16 genomic contig, | 00800 600 C22 |
| | | reference assembly | |
| N/A | gi\|51474257 | Homo sapiens chromosome 17 genomic contig, | 00800 538 106 |
| | | reference assembly | |
| N/A | gi\|51475307 | Homo sapiens chromosome 21 genomic contig, | 00800 597 N16 |
| | | reference assembly | |
| N/A | gi\|52138581 | Homo sapiens pim-3 oncogene (PIM3), mRNA | 00800 586 G15 |
| N/A | gi\|52632376 | Homo sapiens melanoma antigen family D, 1 | 00800 550 119 |
| | | (MAGED1), transcript variant 2, mRNA | |
| N/A | gi\|54111426 | Homo sapiens RAB11 family interacting protein 4 (class | 00800 578 P18 |
| | | II) (RAB11 FIP4), mRNA | |
| N/A | gi\|55741844 | Homo sapiens valyl-tRNA synthetase like (VARSL), | 00800 596 F14 |
| | | mRNA | |
| N/A | gi\|55770883 | Homo sapiens ubiquitin associated domain containing 1 | 00800 600 F14 |
| | | (UBAOC1), mRNA | |
| N/A | gi\|55925649 | Homo sapiens transcription elongation factor A (SII)-like | 00800 541 G08 |
| | | 2 (TCEAL2), mRNA | |
| 175 | gi\|56117827 | Homo sapiens speckle-type POZ protein (SPOP), | 00800 574 K08 |
| | | transcript variant 3, mRNA | |
| N/A | gi\|56117829 | Homo sapiens speckle-type POZ protein (SPOP), | 00800 584 M23 |
| | | transcript variant 4, m RNA | |
| N/A | gi\|57242791 | Homo sapiens adenomatosis polyposis coli 2 (APC2), | 00800 532 G10 |
| | | mRNA | |
| N/A | gi\|57617038 | Homo sapiens tubulin tyrosine ligase-like family, | 00800 589 A07 |
| | | member 12 (TILL 12), mRNA | |
| N/A | gi\|5802969 | Homo sapiens AFG3 ATPase family gene 3-like 2 | 00800 529 K21 |
| | | (yeast) (AFG3L2), nuclear gene encoding mitochondrial | |
| | | protein, mRNA | |
| N/A | gi\|58530844 | Homo sapiens zyxin (ZYX), transcript variant 2, mRNA | 00800 546 G19 |
| N/A | gi\|5902121 | Homo sapiens spectrin, beta, non-erythrocytic 2 | 00800 541H2O |
| | | (SPTBN2), mRNA | |
| N/A | gi\|5902157 | Homo sapiens ring finger protein 113A (RNF113A), | 00800 525 E17 |
| | | mRNA | |
| N/A | gi\|60498971 | Homo sapiens 3-phosphoinositide dependent protein | 00800 584 017 |
| | | kinase-1 (POPK1), transcript variant 1, mRNA | |
| N/A | gi\|61102726 | Homo sapiens La ribonucleoprotein domain family, | 00800 556 H18 |
| | | member 1 (LARP1), transcript variant 1, mRNA | |
| N/A | gi\|62750346 | Homo sapiens histone deacetylase 5 (HOAC5), | 00800 550 B21 |
| | | transcript variant 1, mRNA | |
| N/A | gi\|63082031 | Homo sapiens p53-associated parkin-like cytoplasmic | 00800 526 A18 |
| | | protein (PARC), mRNA | |
| N/A | gi\|63252907 | Homo sapiens IQ motif and WO repeats 1 (IQW01), | 00800 506 F21 |
| | | transcript variant 1, m RNA | |
| N/A | gi\|63497678 | Homo sapiens chromosome 1 open reading frame 131 | 00800 520 P24 |
| | | (C1orf131), mRNA | |
| N/A | gi\|65301138 | Homo sapiens ATPase, Class II, type 9A (ATP9A), | 00800 582 011 |
| | | mRNA | |
| N/A | gi\|65787264 | Homo sapiens lipopolysaccharide-induced TNF factor | 00800 508 010 |
| | | (LITAF), mRNA | |
| N/A | gi\|66346709 | Homo sapiens membrane associated guanylate kinase, | 00800 545 124 |
| | | WW and POZ domain containing 2 (MAGI2), mRNA | |
| N/A | gi\|66348107 | Homo sapiens zinc finger protein 12 (ZNF12), mRNA | 00800 581 L20 |
| N/A | gi\|66879658 | Homo sapiens AOP-ribosylation factor 1 (ARF1), | 00800 552 019 |
| | | transcript variant 4, mRNA | |
| N/A | gi\|6912325 | Homo sapiens family with sequence similarity 50, | 00800 564 004 |
| | | member B (FAM50B), mRNA | |
| N/A | gi\|70609888 | Homo sapiens ribosomal protein S3A (RPS3A), mRNA | 00800 550 007 |
| N/A | gi\|7262387 | Homo sapiens asparaginyl-tRNA synthetase (NARS), | 00800 528 H03 |
| | | mRNA | |
| N/A | gi\|74027246 | Homo sapiens polyglutamine binding protein 1 | 00800 591 P06 |
| | | (PQBP1), transcript variant 2, mRNA | |
| N/A | gi\|7657670 | Homo sapiens upstream binding transcription factor, | 00800 530 G08 |
| | | RNA polymerase I (UBTF), mRNA | |
| N/A | gi\|7669552 | Homo sapiens valosin-containing protein (VCP), mRNA | 00800 533 A21 |
| N/A | gi\|7705400 | Homo sapiens HOCMA18P protein (HOCMA18P), | 00800 518 022 |
| | | mRNA | |
| N/A | gi\|7706556 | Homo sapiens chromosome 9 open reading frame 78 | 00800 562 K11 |
| | | (C90rf78), transcript variant 2, mRNA | |
| N/A | gi\|77628146 | Homo sapiens endoplasmic reticulum protein 29 | 00800 580 J04 |
| | | (ERP29), transcript variant 1, mRNA | |
| N/A | gi\|77917603 | Homo sapiens ubiquitin-binding protein homolog | 00800 569 P20 |
| | | (UBPH), mRNA | |
| N/A | gi\|8051607 | Homo sapiens heme oxygenase (decycling) 2 (HMOX2), | 00800 528 017 |
| | | mRNA | |
| N/A | gi\|83716023 | Homo sapiens kinesin family member 21 B (KIF21 B), | 00800 528 P18 |
| | | mRNA | |
| N/A | gi\|83776595 | Homo sapiens CaM kinase-like vesicle-associated | 00800 512 F08 |
| | | (CAMKV), mRNA | |
| N/A | gi\|87578395 | Homo sapiens microtubule-associated protein 2 | 00800 580 L07 |
| | | (MAP2), transcript variant 1, m RNA | |
| N/A | gi\|88942318 | Homo sapiens chromosome 1 genomic contig, | 00800 568 K22 |
| | | reference assembly | |
| N/A | gi\|88942921 | Homo sapiens chromosome 1 genomic contig, | 00800 523 H16 |
| | | reference assembly | |
| N/A | gi\|88955854 | Homo sapiens chromosome 2 genomic contig, alternate | 00800 540 D18 |
| | | assembly (based on Celera assembly) | |
| N/A | gi\|88999178 | Homo sapiens chromosome 6 genomic contig, alternate | 00800 544 J14 |
| | | assembly (based on Cetera assembly) | |
| N/A | gi\|88999564 | Homo sapiens chromosome 6 genomic contig, alternate | 00800 587 F02 |
| | | assembly (based on Cetera assembly) | |
| N/A | gi\|89028628 | Homo sapiens chromosome 8 genomic contig, alternate | 00800 603 N12 |
| | | assembly (based on Cetera assembly) | |
| N/A | gi\|89037929 | Homo sapiens chromosome 14 genomic contig, | 00800 581 N05 |
| | | alternate assembly (based on Cetera assembly) | |
| N/A | gi\|89057698 | Homo sapiens chromosome 19 genomic contig, | 00800 566 M21 |
| | | alternate assembly (based on Cetera assembly) | |
| N/A | gi\|89059606 | Homo sapiens chromosome X genomic contig, | 00800 516 G22 |
| | | reference assembly | |
| N/A | gi\|89060486 | Homo sapiens chromosome X genomic contig, | 00800 538 J13 |
| | | reference assembly | |
| N/A | gi\|8922357 | Homo sapiens PRP38 pre-mRNA processing factor 38 | 00800 580 H16 |
| | | (yeast) domain containing B (PRPF38B), mRNA | |
| N/A | gi\|90652860 | Homo sapiens protein tyrosine phosphatase, non- | 00800 583 A01 |
| | | receptor type 5 (striatum-enriched) (PTPN5), transcript | |
| | | variant 3, mRNA | |
| N/A | gi\|90903237 | Homo sapiens glutathione peroxidase 4 (phospholipid | 00800 560 E13 |
| | | hydroperoxidase) (GPX4), transcript variant 2, mRNA | |
| N/A | gi\|94536841 | Homo sapiens ribose 5-phosphate isomerase A (ribose | 00800 525 P07 |
| | | 5-phosphate epimerase) (RPIA), mRNA | |
| N/A | gi\|94538369 | Homo sapiens zuotin related factor 1 (ZRF1), mRNA | 00800 582 P15 |

SEQUENCE OF Homo sapiens speckle-type POZ protein (SPOP), transcript variant 3, mRNA from Genbank Accession No. gi:56117827 (SEQ ID NO: 175):

```
      1 ggggaggagg ccgcgcgggg tggggtctgg cggtacgcgc tggctgcgtc gacgtgctga
    61 cgccatgacg ccccggctgg tgtgtgtcgg tgtgtatgtg tgtgtgtgag tgtgcgcgct
  121 ccgagtgtgt gtgtatttgt gtatcggcgg tcccgcaggt cccggatgtt gcggacagta
  181 tgaggcaagc gcagggggac ggggaccagc agctgtcgcc gccgctctca ggctctggga
  241 accacccttc tactttctgt ctctaggaat ttcactactc tagggtgaag agggaacaga
  301 aatctttgcc ccctgacttt ggaaatctcg tttaaccttc aaactggcga tgtcaagggt
  361 tccaagtcct ccacctccgg cagaaatgtc gagtggcccc gtagctgaga gttggtgcta
  421 cacacagatc aaggtagtga aattctccta catgtggacc atcaataact ttagcttttg
  481 ccgggaggaa atgggtgaag tcattaaaag ttctacattt tcatcaggag caaatgataa
  541 actgaaatgg tgtttgcgag taaaccccaa agggttagat gaagaaagca aagattacct
  601 gtcactttac ctgttactgg tcagctgtcc aaagagtgaa gttcgggcaa aattcaaatt
  661 ctccatcctg aatgccaagg gagaagaaac caaagctatg gagagtcaac gggcatatag
  721 gtttgtgcaa ggcaaagact ggggattcaa gaaattcatc cgtagagatt ttcttttgga
  781 tgaggccaac gggcttctcc ctgatgacaa gcttaccctc ttctgcgagg tgagtgttgt
  841 gcaagattct gtcaacattt ctggccagaa taccatgaac atggtaaagg ttcctgagtg
  901 ccggctggca gatgagttag gaggactgtg ggagaattcc cggttcacag actgctgctt
  961 gtgtgttgcc ggccaggaat tccaggctca caaggctatc ttagcagctc gttctccggt
1021 ttttagtgcc atgtttgaac atgaaatgga ggagagcaaa aagaatcgag ttgaaatcaa
1081 tgatgtggag cctgaagttt ttaaggaaat gatgtgcttc atttacacgg ggaaggctcc
1141 aaacctcgac aaaatggctg atgatttgct ggcagctgct gacaagtatg ccctggagcg
1201 cttaaaggtc atgtgtgagg atgccctctg cagtaacctg tccgtggaga acgctgcaga
1261 aattctcatc ctggccgacc tccacagtgc agatcagttg aaaactcagg cagtggattt
1321 catcaactat catgcttcgg atgtcttgga gacctctggg tggaagtcaa tggtggtgtc
1381 acatccccac ttggtggctg aggcataccg ctctctggct tcagcacagt gcccttttct
1441 gggaccccca cgcaaacgcc tgaagcaatc ctaagatcct gcttgttgta agactccgtt
1501 taatttccag aagcagcagc cactgttgct gccactgacc accaggtaga cagcgcaatc
1561 tgtggagctt ttactctgtt gtgaggggaa gagactgcat tgtggcccca gacttttaaa
1621 acagcactaa ataacttggg ggaaacgggg ggagggaaaa tgaaatgaaa accctgttgc
1681 tgcgtcactg tgttcccttt ggcctggctg agtttgatac tgtggggatt cagtttaggc
1741 gctggcccga ggatatccca gcggtggtac ttcggagaca cctgtctgca tctgactgag
1801 cagaacaaat cgtcaggtgc ctggagcaaa aaggaaaaaa aaaaaagaaa ggacattgag
1861 ttttaacaga agggaaaagg aaagaagaaa agatttttgc agaatttctc aaaaatcagt
1921 ttgtggattc cagtagtatt tatattgaga gaaacaaatt ttagtccttc taactgtgct
1981 aaaacttgga tatttgtgaa aactccttac caccatacaa gcatcagaag agctctcttg
2041 ttgttagcac ttattgtttg caagaacaga atacatcctt ttatcctttt atgaaaaatg
2101 acaagtgaag gcaaaagggg aaggttattt gatctggaag atgagtgttc tgatgtggtg
2161 gcttttgcaa aaatctttat tggtgttgaa aactggaaaa aataactcat ccagaattca
2221 tattgtcttg acaagaacta tggttctctg tttttagata ttgtggaaaa tgtttttggg
2281 catttttctc tgattttatt tcttctcccc cacccctttt tctaaaaaac aaacaaaaaa
2341 aaaaacacac aaaacaaaaa cagaacaaaa gaagagagaa ggaaatttta tcaattaaaa
2401 atgctgtgtg ataaaatccc agcccagatt gctcagctgt ttgtacctga cttgccgcct
2461 gcataggagc cagttctgtt ccttctgact agcccctctt cctccagggg agaacttcca
2521 aatgttaatt tttttttttt tgaaaatata aataattact attttgtact gtgtggtatc
2581 tctggtcttt tgtttcactc acctgccttg tctcttgggt ctgagtccct tgcttaaggg
2641 attttgaagt cctagttttc agcttgcaga gattatgtct gaaatgccta atgagtcgca
2701 gggatttgtt gagactccgt aatctcaagt tctctttgtg agctatcagc atctgccagt
2761 ctcttgtcct ccctgagtat ctcacagtcc atatcctgat gagggatcag gcccctacct
2821 ctgccaaggc aagtaatggt agtgggcttt taaactgccc cccgtatgtt ttaagaccta
2881 atccccacct cccttcttct aactaaatat aaaaagatcc aggggacata aatgtggaga
2941 ttaaataaag ggaaattatt gtctctaaaa aaaaaaaaaa aa
```

1. Method for diagnosing inflammatory prostate diseases or prostate carcinoma, wherein
a) at least one marker sequence of a protein encoded by the cDNA complimentary to an mRNA having the sequence set forth in SEQ ID NO: 175 and/or an mRNA encoding full length speckle-type POZ protein (SPOP) is applied to a solid support and
b) is brought into contact with body fluid or tissue extract of a human patient and
c) the detection of an interaction of the body fluid or tissue extract with the marker sequences from a) is carried out, and
d) comparing the degree of interaction of the marker sequences from a) with the body fluid or tissue extract of the human patient with the degree of interaction of the marker sequences from a) with a body fluid or tissue extract from a human free of inflammatory prostate disease and prostate carcinoma, wherein increased degree of interaction is utilized for diagnosis of inflammatory prostate diseases or prostate carcinoma.

2. Method for the stratification, in particular risk stratification or therapy control of a patient with inflammatory prostate diseases, prostate carcinoma, wherein at least one marker sequence of a cDNA having the sequence set forth in SEQ ID NO: 127 or an mRNA having the sequence set forth in SEQ ID NO: 175 or respectively a protein encoded by a cDNA complimentary to the mRNA having the sequence set forth in SEQ ID NO: 175 or respectively a partial sequence or fragment thereof is determined on or from a patient to be examined.

3. Method according to claim 2, wherein the stratification or the therapy control covers decisions for the treatment and therapy of the patient, in particular the hospitalization of the patient, the use, effect and/or dosage of one or more drugs, a therapeutic measure, or the monitoring of a course of the disease and the course of therapy, etiology, or classification of a disease together with prognosis.

4. Arrangement of marker sequences containing at least one marker sequence of a cDNA having the sequence set forth in SEQ ID NO: 127 or an mRNA having the sequence set forth in SEQ ID NO: 175 or respectively a protein encoded by the cDNA or the mRNA.

5. Arrangement according to claim 4, characterized in that at least 2 to 5 or 10, preferably 30 to 50 marker sequences or 50 to 100 or more marker sequences are contained.

6. Arrangement according to claim 4, characterized in that the marker sequences are present as clones.

7. Assay, protein biochip comprising an arrangement according to claim 4, characterized in that the marker sequences are applied to a solid support.


SEQUENCE LISTING (175 sequences)

SEQ ID NO: 1 - DNA, 255 bp, Homo Sapiens
gaatcgtcgacccacgcgttccgctcgcaggcaccatgactcctgtgaggatgcagcactccctggcaggtcagacctatgccgtgcccttcatccagccagacctgcggcgagaggaggccgtccagcagatggcggatgccctgcagtacctgcagaaggtctctggagacatcttcagcaggtgttctccagtgccaagtaccctgctccagggcgcctgcaggaatatggctccatcttcacgggcgccca

SEQ ID NO: 2 - DNA, 255 bp, Homo sapiens
gaatcgtcgacccacgcgtccgcctgtggctacctctggccctgcgtattccccaggcccagcctcgccaggccaggcctattcagctgctcccccctcctcctgcgccccctcctcctcctcttcttctgaatggcaggaaccagtcctggagcccttcgatccctttgagctggggcagggcagttctccagagcctgagctgctgcgttctcaggagcccagagcagtggggacacctgggcctggaccccg

SEQ ID NO: 3 - DNA, 255 bp, Homo sapiens
gaatcgtcgacccacgcgtccgcctcccctgagctcgagtcttcggatgactcctacgtgtccgctggagaagagcccctagaggcccctgtgtttgagatccccctgcagaatgtggtggtggcaccaggggcagatgtgctgctcaagtgtatcatcactgccaaccccccgccccaagtgtcctggcacaaggatgggtcagcgctgcgcagcgagggccgcctcctcctccgggctgagggtgagcggcac

SEQ ID NO: 4 - DNA, 255 bp, Homo sapiens
gaatcgtcgacccacgcgtccgcggacgcgtgggttgcagtggagcccgcagagaacgtgagcgaagccctccgagctgtgcccagccccacgacgggagaggagggctctgtgcacagcagggaggcaaaagagagttctgcagcccaagctggctctcatgccacgcaccctggcacctcggttcttgagggaggagctgcaggatccatgtctcccagcagagtggaagccaatgtccccatggttcagcct

SEQ ID NO: 5 - DNA, 255 bp, Homo sapiens
gaatcgtcgacccacgcgtccggaagcggcacaccgtctcgccggacacgcgtctctggagcggtacgaaggcctggcgcacagcagcagcaagggccgggaagcccctgggcggcctccccgggctgtacccaagctgggtgtgccaccctccagccccacacacggtccagctcccgcctgtaggagcggcgcagccaaggctgtgggggcccccaagccccctgttggtggaggcaagggccgtggcctagt

SEQ ID NO: 6 - DNA, 255 bp, Homo sapiens
gaatcgtcgacccacgcgtccgaaaggcgcggcggggaggggagccggggccccagccctgccgggaaacgggttcccagtcccggccgagagcgcgggcgccgaggggaccgacccggactccccgcaaccccccaaccgccaggccatggcgcccacggggctcctggtggccggcgcctccttcctcgcgttccgggggctgcactgggggttgcggcggctgcccacgccggaatcggccgctcgggaccg

SEQ ID NO: 7 - DNA, 255 bp, Homo sapiens
gaatcgtcgacccacgcgtccgggctgcctggaggcagatgctcagccagcattgggcagcccccgcttcctgctcccctacccccttcacatggcagtagttctgggcaccccagcaaaccatattatgctccaggggcgcccactccaagacccctccatgggaagctggaatccctgcatggctgtgtgcaggcattgctccgggagccagcccagccagggctttgggaacagcttgggcaactgtacgag

SEQ ID NO: 8 - DNA, 255 bp, Homo sapiens
  misc_feature (34)..(34): n is a, c, g, or t
gaatcgtcgacccacgcgtccggacggacccagntctcggcctgggtggacctcccaacccaccagggccaagcccaaaagccctggccaagatgatgggcggcgcaggtcctggcagctcactggaagcccgcagcccctcggaccttcacatctcacccttggccaagaagttgccaccaccaccgggcagccccctgggccactcaccaactgcctctcctcctcctacggcccgaaagatgttcccaggcc

SEQ ID NO: 9 - DNA, 255 bp, Homo sapiens
gaatcgtcgacccacgcgtccgggtgtgttcaccggatcagagatagcagagcgccgagttggggccacgaaggcgtgaggggagtcgtcgtccctcctgcacgaaagcgtctaagccttggcgacgccgccctgggggacccacgtcaggcctgggatagggaccgctgtccccgggtccctaccaatgtcgcccgtcgctcccggcccagctctacccgcagagtctgatggcagcggccactctgaggacgc

SEQ ID NO: 10 - DNA, 255 bp, Homo sapiens
gaatcgtcgacccacgcgtccgagctacccaagcgcctcagccttgacagctcctcctccctcgagtctcttgcttctgctcagtctgtttccaacgccctgcccttgggttaccagcaaccccccttctctcccaccggtgcggacagcatcgcctcagatgccatctctgtgtacagtctgagctccattgcctcctcaatgagctttgtctccaaacccgagggtggatcagagggtggaggccccggagga

SEQ ID NO: 11 - DNA, 255 bp, Homo sapiens
gaatcgtcgacccacgcgtccgggtcacggcaatcaaccccctgctgtggttcccgaaccccaaggcccgatgggtcccgcgggggtcgcggcgaggccagggcgctttttcggcgtctacctgctctactgcctgaacccccggtaccggggccgcgtctacgtggggttcactgtcaacactgctcgtcgggtccagcagcacaatgggggccgcaaaaaaggcggggcctggcggaccagcgggcgagggcc

SEQ ID NO: 12 - DNA, 255 bp, Homo sapiens
gaatcgtcgacccacgcgtccgagaaaacgcggaacagggcctctgtggcaaatggaggcgagaaggcctcagagaaactcgccccagaagaagttcccctaagcgctgaggcccaggcacaacagttggcccaggaattggcttggtgtgtggagcaactggagctgggcctcaagaggcagaaacccaccccgaaacagaaagagcaggctattggagcaatccgaaccctgcgcagcaaaagaacgcccttg

SEQ ID NO: 13 - DNA, 182 bp, Homo sapiens
  misc_feature (4)..(4): n is a, c, g, or t
atanaatcggtcgacccacgcgtccgggnagnntgtnagcngtngncgnctgccaanatgccaccccnggngtcgggttgcgccatgctgcgagcagcgagtgagcctgtaccanccnnnctctgtagtcttntgagnaggattccactcctgtgtcctgtgcgtacnaanatgaancggtn

SEQ ID NO: 14 - DNA, 255 bp, Homo sapiens
gaatcgtcgacccacgcgtccgcccacctttgcagatatctgcacctcaggagagactgtattccacgtggattgggggctccatccttgcctccctggacacctttaagaagatgtgggtctccaaaaaggaatatgaggaagacggtgcccgatccatccacagaaaaaccttctaatgtcgggacatcatcttcacctctctctgaagttaactccactttaaaactcgctttcttgagtcggagtgtttgc

SEQ ID NO: 15 - DNA, 255 bp, Homo sapiens
gaatcgtcgacccacgcgtccgcccacgcgtccggatggatgaagccccaaattcagccccacccacagagaagccttcctacactcagcctctgtccacccttggcaaatctttcaagctctctcctccaggaaagtggggccccaactcagtcactccacccccttccaggtccctgaggctggttctactgtatccccatcacctccacaactccactcacccctgacggctccatccacctcaccagttgg

SEQ ID NO: 16 - DNA, 255 bp, Homo sapiens
  misc_feature (9)..(9): n is a, c, g, or t
aaatcgtcnacccacncntccncgatggnggatacnctggagtccatcgctggangaccnacatgcggagcatttgtgcgagtnttggagaagacgggatggnacagntgctaccacnacngcantatanntccggtggngtgngttgcgttgtgtgcnacnctgccattgncctttctaaatacctgaaaaacgcccgagttttctggcgacgggnaccacgcgctgagccggcggncgntgntggagcngggt

SEQ ID NO: 17 - DNA, 255 bp, Homo sapiens
gaatcgtcgacccacgcgtccggcacaacccatggctgcgctgggctgcgcgaggctgaggtgggcgctgcgaggggccggccgtggcctctgcccccacggggccagagccaaggccgcgatccctgccgccctcccctcggacaaggccaccggagctcccggagccgggcctggtgtccggcggcggcaacggagcttagaggagattccacgtctaggacagctgcgcttcttctttcagctgttcgttca

SEQ ID NO: 18 - DNA, 255 bp, Homo sapiens
gaatcgtcgacccacgcgtccgcggacgcgtgggcggacgcgtgggcggacgcgtgggctggtgggggtcggggaggccagttattccaccatcgcgcccactctcattgccgacctctttgtggccgaccagcggagccggatgctcagcatcttctactttgccattccggtgggcagtggtctgggctacattgcaggctccaaagtgaaggatatggctggagactggcactgggctctgagggtgacacc

SEQ ID NO: 19 - DNA, 255 bp, Homo sapiens
gaatcgtcgacccacgcgtccgccaagatcaacgcgagcaagaatcagcaggatgacggtaaaatgtttattggaggcttgagctgggatacaagcaaaaaagatctgacagagtacttgtctcgatttggggaagttgtagactgcacaattaaaacagatccagtcactgggagatcaagaggatttggatttgtgcttttcaaagatgctgctagtgttgataaggttttggaactgaaagaacacaaactg

SEQ ID NO: 20 - DNA, 255 bp, Homo sapiens
gaatcgtcgacccacgcgtccgaccgaagtggtcgaactgctcgagctaccaatgaaggcctcagtctgatgctcattgggcctgaggatgtgatcaactttaagaagatttacaaaacgctcaagaaagatgaggatatcccactgttccccgtgcagacaaaatacatggatgtggtcaaggagcgaatccgtttagctcgacagattgagaaatctgagtatcggaacttccaggcttgcctgcacaactct

SEQ ID NO: 21 - DNA, 255 bp, Homo Sapiens
gaatcgtcgacccacgcgtccgcccacgcgtccggacgaggctgcggtgtctgctgctattctccgagcttcgcaatgccgcctaaggacgacaagaagaagaaggacgctggaaagtcggccaagaaagacaaagacccagtgaacaaatccgggggcaaggccaaaaagaagaagtggtccaaaggcaaagttcgggacaagctcaataacttagtcttgtttgacaaagctacctatgataaactctgtaag

SEQ ID NO: 22 - DNA, 255 bp, Homo Sapiens
gaatcgtcgacccacgcgtccgaggtgagaggcgcctctgcccagctgcccctactgggaagtgaggagcccctctgcccggctagccgccccgtccaggagggaggtgggggggtcagccccccacccagccagccgccttgtccgggagggaggtggggtgatcagccccccacctggccagccaccccatccgggagggaggtggggtggttaaccccccgccaggccagccgccccctccaggagggaggt

SEQ ID NO: 23 - DNA, 255 bp, Homo Sapiens
gaatcgtcgacccacgcgtccggcggcgaacgcggagagcacgccatgaaggcttcgggcacgctacgagagtacaaggtagtgggtcgctgcctgcccacccccaaatgccacacgccgcccctctaccgcatgcgaatctttgcgcctaatcatgtcgtcgccaagtcccgcttctggtactttgtatctcagttaaagaagatgaagaagtcttcaggggagattgtctactgtgggcaggtgtttgagaag

SEQ ID NO: 24 - DNA, 255 bp, Homo Sapiens
gaatcgtcgacccacgcgtccggggtgaggccctcacttcatccggcgactagcaccgcgtccggcagcgccagccctacactcgcccgcgccatggcctctgtctccgagctcgcctgcatctactcggccctcattctgcacgacgatgaggtgacagtcacggaggataagatcaatgccctcattaaagcagccggtgtaaatgttgagcctttttggcctggcttgtttgcaaaggccctggccaacgtc

SEQ ID NO: 25 - DNA, 255 bp, Homo Sapiens
gaatcgtcgacccacgcgtccgcccacgcgtccgaaccgccgccaggtcgctgttggtccacgccgcccgtcgcgccgcccgcccgctcagcgtccgccgccgccatgggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatg

SEQ ID NO: 26 - DNA, 255 bp, Homo Sapiens
gaatcgtcgacccacgcgtccgcgccgcccaagatgccgaaaggaaagaaggccaagggaaagaaggtggctccggccccagctgtcgtgaagaagcaggaggctaagaaagtggtgaatcccctgtttgagaaaaggcctaagaattttggcattggacaggacatccagcccaaaagagacctcacccgctttgtgaaatggccccgctatatcaggttgcagcggcagagagccatcctctataagcggctg

SEQ ID NO: 27 - DNA, 255 bp, Homo Sapiens
gaatcgtcgacccacgcgtccggaggtgcaccatccatcacatctgtcacaccaagaggactgtgcagagatgaggaagacacctcttttgaatcactttctaaattcaatgtcaagtttccacctatggacaatgactcaactttcttacatagcactccagagagacccggcatccttagtcctgccacgtctgaggcagtgtgccaagagaaatttaatatggagttcagagacaacccagggaactttgtt

SEQ ID NO: 28 - DNA, 255 bp, Homo Sapiens
  misc_feature (1)..(1): n is a, c, g, or t
naatcgtcgacccacgcgtccggaggcggcggcggcggcggcgggagcgggagcgggcggcgagtggggagcggggccggnagtggagcagccgccgcggcgggactggaccgagcctcgccggcgcgcacctgcccgcagcgcccgcggagcgcgcagcgcggcccgagcgcgacgacctgccgagcgncggccgaggcggcggtgtgggcgcgtcaggccgcgacgagggcgctgagacnaatttacatgtat

SEQ ID NO: 29 - DNA, 255 bp, Homo Sapiens
gaatcgtcgacccacgcgtccgggaatcctgatatcccaaatccagctctggctcaaagagaagagcaaaaaaagattgatggagctgaacctcttacaccagaagagactgaagaaaaggaaaaacttctcacacaaggtttcacaaactggactaaacgagattttaaccagtttattaaagctaatgagaaatatggaagagatgacattgataacatagctcgagaggtagagggcaaatcccctgaggag

SEQ ID NO: 30 - DNA, 255 bp, Homo Sapiens
gaatcgtcgacccacgcgtccgcccacgcgtccggcggccgcgtcgggaccggccgcgatgggaccggaggcggcgcgggcggcggcggcgcagacaaaggggccaaaccagtgctcctgccacctctctggctgccccctagagcctgcccatcccagcctgaccaatgtccacagccagggagcagccaatcttcagcacacgggcgcacgtgttccaaattgacccagccaccaagcgaaactggatcccag

SEQ ID NO: 31 - DNA, 255 bp, Homo Sapiens
gaatcgtcgacccacgcgtccggccccaggcaccgccagagtgtcctgctacctttgtctcacgattccagtgcagtagtgccaaaaggccccttcccacgtccctcgagctctgtagcctggtctgtgcattgtggctgtcaaatccatgtgtcccccctgtggtcgtgtgccccatggagaccatcaaggtgaagttcatccacgaccagacctccccaaaccccaagtacagaggattcttccacggggtta

SEQ ID NO: 32 - DNA, 255 bp, Homo Sapiens
gaatcgtcgacccacgcgtccggcctgtgcccagatcctgggagaaccccagccgagcccagcctagcccgagcccagcccgagcggagccggagccccaagcccgagccgcgcccagcccgagcagagccctccagccgctcaccccgcgtgccaccccagcgaccctcagccgctctctgcccttctctcggccccgcgcccgccctcgcggcccctctgcccaatgaaactggccgcgatgatcaagaagat

SEQ ID NO: 33 - DNA, 255 bp, Homo Sapiens
gaatcgtcgacccacgcgtccgagcagcttgaacaagtcctgtcaaaaaaactggaggaggttgaaggtgaaatatccagttaccaggatgcaattgagattgaactagaaaacagccggccaaaggccaaggggggcctggccagcctggcaaaagatggctccaccgaggacaccgcgtccagcttgtgtggcgaggaggacacagaggacgaggagctggaagccgcggccagccacctgaacaaagactta

SEQ ID NO: 34 - DNA, 255 bp, Homo Sapiens
gaatcgtcgacccacgcgtccgcagacggcgcggccgaacccagcgggtgccgcttctccacccgaggcttccacctccaacgagccatgttccaggctgcaggagccgcccaggccaccccctctcatgacgccaaaggcggcggcagcagcacggtgcagcgctccaagtccttcagcctgcgggcccaggtgaaggagacctgcgccgcctgccagaagaccgtgtaccccatggagcggctggtggccgac

SEQ ID NO: 35 - DNA, 255 bp, Homo Sapiens
gaatcgtcgacccacgcgtccgggagggggtcgcgcgaagtgccagatgcaggcggggaagcccatcctctattcctatttccgaagctcctgctcatggagagttcgaattgctctggccttgaaaggcatcgactacgagacggtgcccatcaatctcataaaggatgggggccaacagttttctaaggacttccaggcactgaatcctatgaagcaggtgccaaccctgaagattgatggaatcaccattca

SEQ ID NO: 36 - DNA, 255 bp, Homo Sapiens
gaatcgtcgacccacgcgtccgggaagacctgcggatggacaggagcgggcaggcccgcacatatccacttgctggagcccatgtttacagacagggacatacaccatgcagatcctgagttcctgctgtatgagcagggatatccatgcttatgtatccaaacacagagacccatgggaacaaatgagacacatatagatactgagacctgtgtgtacagtaggaccatgcactcacacccatctggagaggga

SEQ ID NO: 37 - DNA, 255 bp, Homo Sapiens
gaatcgtcgacccacgcgtccgcccacgcgtccgcccacgcgtccggtggaggccagcatcctcgcggagctggccaggcgagcctcgggacccatttgtctgctgttggccagcctgctgtcgctggtctcagctgtcagtgcctgccgagtgttcaagctctggcccctgtccttcctcgggagcaaactctccacgtgcgaacagctccggcaccggctggagcacctcacgctaatcttcagtacacggaa

SEQ ID NO: 38 - DNA, 255 bp, Homo Sapiens
gaatcgtcgacccacgcgtccggttctaagagggacatggcctccaatgaaggcagtcgcatcagtgctgcctctgtggccagcactgccattgctgcggcccagtgggccatctcacaggcctcccaaggtggggagggtacctgggccacctccgaggagccgccggtcgactacagctactaccaacaggatgagggctatggcaacagccagggcacagagtcttccctctatgcccatggctacctcaag

SEQ ID NO: 39 - DNA, 255 bp, Homo Sapiens
gaatcgtcgacccacgcgtccgggcctcaacctgaaggtggtgcagcttttccgcgacccgagggcggtgcacaactcgcgcctcaagtctaggcagggactgctgcgcgagagcatccaggtgctgcgcacccgccagaggggcgaccgcttccaccgtgtgctgctggcgcacggcgtgggtgctcgccccgggggccagtctcgcgcgctgcccgccgcgccgcgcgccgatttcttcctgaccggtgcgct

SEQ ID NO: 40 - DNA, 255 bp, Homo Sapiens
gaatcgtcgacccacgcgtccgcccacgcgtccgcccacgcgttccgcccacgcgtccggccggcgtcggggagcggcggtaccgggcggctgcggggctggctcgacccagctggaggtctcggcgtccgcgtcctgcggtgccctgggacccgccgacatgaatcccatcgtagtggtccacggcggcggagccggtcccatctccaaggatcggaaggagcgagtgcaccagggcatggtcagagccgccac

SEQ ID NO: 41 - DNA, 255 bp, Homo Sapiens
gaatcgtcgacccacgcgtccggagagaaaggaaagcgcgaggagccgccgccaccaccagcgcagcagtcctggagctgtgaggagattcgggccgtcaccctgcctcccctgcgtcccgccaccggccgcttctgtcctcggacccattccaacaatctcgtaaaacatggtggattactatgaagttctaggcgtgcagagacatgcctcacccgaggatattaaaaaggcatatcggaaactggcactgaa

SEQ ID NO: 42 - DNA, 255 bp, Homo Sapiens
  misc_feature (108)..(108): n is a, c, g, or t
gaatcgtcgacccacgcgtccgcggacgcgtgggccaccatctctagttcttcttccactaaaggaataaagaggaaagctacagaaattagcactgcagtggttcanaggtcagctaccattggcagttctccagttctctatagccagtcagctatagctacaggtcaccaggcagcagggattggaaaccaggcaacaggaattggacatcagacaataccagttagccttccagcagcaggaatgggtcat

SEQ ID NO: 43 - DNA, 255 bp, Homo Sapiens
gaatcgtcgacccacgcgtccgcgcgggagaggagccccactcccccagcgccgcagccaccgcagccaccgcagcccgtgcgccccgcgccctcgagcgccatggccaaggaaggcgtggagaaggcggaggagacggagcaaatgatcgagaaggaggcaggcaaggagccggcggagggcggcggcggcgacggctcgcaccgcctcggggacgcccaggagatgcgcgcggtggtgctggctggcttcggg

SEQ ID NO: 44 - DNA, 255 bp, Homo Sapiens
gaatcgtcgacccacgcgtccgggggtctcagagcagaccacctgccaggaagtggtcatcgcactagcccaagcaataggccagactggccgctttgtgcttgtgcagcggcttcgggagaaggagcggcagttgctgccacaagagtgtccagtgggcgcccaggccacctgcggacagtttgccagcgatgtccagtttgtcctgaggcgcacagggcccagcctagctgggaggccctcctcagacagctg

SEQ ID NO: 45 - DNA, 255 bp, Homo Sapiens
gaatcgtcgacccacgcgtccgaaaggcgaccctcgaaagataagattgaagatgagctggagatgaccatggtttgccatcggcccgagggactggagcagctcgaggcccagaccaacttcaccaagagggagctgcaggtcctttatcgaggcttcaaaaatgagtgccccagtggtgtggtcaacgaagacacattcaagcagatctatgctcagtttttccctcatggagatgccagcacgtatgcccat

SEQ ID NO: 46 - DNA, 255 bp, Homo Sapiens
gaatcgtcgacccacgcgtccgggccgctgtacatgagcaccaagaacaccatactgaaagcctacgatgggcgtttcaaggacatcttccaggagatctttgacaagcactataagaccgacttcgacaagaataagatctggtatgagcaccggctcattgatgacatggtggctcaggtcctcaagtcttcgggtggctttgtgtgggcctgcaagaactatgacggagatgtgcagtcagacatcctggcc

SEQ ID NO: 47 - DNA, 255 bp, Homo Sapiens
gaatcgtcgacccacgcgtccgcttttagcaccagtgtgggaaatgaggacgccaggacagcctggcccgaattacaacagagccatgctgttaatcagctcaaagatttgttgcgccaacaagcagataaggaaagtgaagtatctccgtcaagaagaagaaaaatgtcccccttgaggtcattagaacatgaggaaaccaatatgcctactatgcacgaccttgttcatactattaatgaccagtctcaatat

SEQ ID NO: 48 - DNA, 255 bp, Homo Sapiens
  misc_feature (1)..(1): n is a, c, g, or t
naatcgtcgacccacgcgtccggantacgggggnaagaactgctcanaagccggccgagtacggctacntgagcctggggcgcctgggggccgccaagggcgcgctgcaccccggntgcttcgcgcccctgcacccaccgnccccgccgccgccgccgcccgccgagctcaaggcggagccgggcttcnagcccgcggactgcaagcggaangaggaggccggggcgccgggcggcgncgcaagnatgncgncgn

SEQ ID NO: 49 - DNA, 255 bp, Homo Sapiens
gaatcgtcgacccacgcgtccgcccacgcgtccgggacgcgtgcggggcaggtcacgcttgggcctggctgcctcaggagccaccctgcacggcgccgggcgaggctgtgcgcgccctcccggaccctccactttggggctttgcggtgtagcggcgcgccccactcccttacgtgttcagtcccgaatcacgtacgggccgctgcagctccaggacggcggacgccagcgctctcggatccccaggcccctccg

SEQ ID NO: 50 - DNA, 255 bp, Homo Sapiens
gaatcgtcgacccacgcgtccgcagggtctccggtaggggaaggcactggatcgcctcccaagtggcagattggtgagcaggaatttgaagccctcatgcggatgctcgataatctgggctacagaactggctacccttatcgataccctgctctgagagagaagtctaaaaaatacagcgatgtggaggttcctgctagtgtcacaggttactcctttgctagtgacggtgattcgggcacttgctccccactc

SEQ ID NO: 51 - DNA, 255 bp, Homo Sapiens
gaatcgtcgacccacgcgtccgctctctcgtccggtgtctgcgtgccaagctgtattaccaactgcttgtggactaaagaggatggacatagaacttccacctctgctgtccctaacctgtttgttccattgaacactaacccaaaagaggtccaggagatgaggaacaagatccgagagcagaatttacaggacattaagacggctggccctcagtcccaggttttgtgtggtgtagtgatggacaggagcctc

SEQ ID NO: 52 - DNA, 255 bp, Homo Sapiens
gaatcgtcgacccacgcgtccgggcggaacaggctaccaagtccgtgctgtttgtgtgtctgggtaacatttgtcgatcacccattgcagaagcagttttcaggaaacttgtaaccgatcaaaacatctcagagaattggagggtagacagcgcggcaacttccgggtatgagatagggaacccccctgactaccgagggcagagctgcatgaagaggcacggcattcccatgagccacgttgcccggcagatta

SEQ ID NO: 53 - DNA, 255 bp, Homo Sapiens
gaatcgtcgacccacgcgtccgctgatttccacctagcaatgcccacggagcaggcagagggcttctacaacagcttcctggagcagctgcgtaaaacatacaggccggagcttatcaaagatggcaagtttggggcctacatgcaggtgcacattcagaatgatgggcctgtgaccatagagctggaatcgccagctcccggcactgctacctctgacccaaagcagctgtcaaagctcgaaaaacagcagcag

SEQ ID NO: 54 - DNA, 255 bp, Homo Sapiens
gaatcgtcgacccacgcgtccgaggccactatggaggaaaacacctatccctccttccttaagtctgatatttatttggaatatacgaggacaggctcggagagccccaaagtctgtagtgaccagagctctgggtcagggacagggaagggcatatctggatacctgccgaccttaaatgaagatgaggaatggaagtgtgaccaggacatggacgaggacgatggcagagacgctgctccccccggaagactc

SEQ ID NO: 55 - DNA, 255 bp, Homo Sapiens
gaatcgtcgacccacgcgtccgagaagactcgccgaaagttcctggacctgggggtcaccctgcgccgagcatccacgggcaagagccggaaggagaaaggcagcaaccgactgtccatgggcagcagggagtcagtggaggggtccggcaggtcagggggctccccgttcctgcctttttcctggttcacggacagcggcaagggctcagcatcctcgggtagcaccacctcccccacctgctcccctaaacac

SEQ ID NO: 56 - DNA, 255 bp, Homo Sapiens
  misc_feature (24)..(24): n is a, c, g, or t
gaatcgtcgacccacgcgtccggncggtccccgggcccactggcggctcctcgggtcgcttcctcagccccgagcagcacgcgtgcagctggcagctcctgctgcccgccccggaggccgcagcgggcagcgagctggcgctgcgctgccanagcccggacggggcgcgccaccagtgcgcctaccgcgggcatccggagcgctgcgcagcctacgccgctcgccgcgcgcacttctggaagcaggtgctgggag

SEQ ID NO: 57 - DNA, 255 bp, Homo Sapiens
gaatcgtcgacccacgcgtccgggccgcgggagacgtggtgccgctgcgggctcgctctgccgtgcgctaggcttggtgggaaggcctgttctcgagtccgcgcttttcgtcaccgccatgtcgggaggtggtgtgattcgtggccccgcagggaacaacgattgccgcatctacgtgggtaacttacctccagacatccgaaccaaggacattgaggacgtgttctacaaatacggcgctatccgcgacatcga

SEQ ID NO: 58 - DNA, 255 bp, Homo Sapiens
gaatcgtcgacccacgcgtccgcccacgcgtccgcactggtgtgtcctccccttccgagtgctgctggtactcacctcacatcgaggaatacagatgtacgagtccaatggctacaccatggtctactggcatgcactggactctggagatgcctccccagtacaggctgtgtttgcccggggaattgctgccagtggccacttcatctgtgtgggaacgtggtcaggccgggtgctggtgtttgacatcccagc

SEQ ID NO: 59 - DNA, 255 bp, Homo Sapiens
gaatcgtcgacccacgcgtccgcccacgcgtccgcggcggcgggagcggtcatggaggcgggcgccggagccggcgcgggagccgcgggctggagctgcccgggcccaggacccacagtgaccactctaggctcctatgaggcttccgagggctgtgagaggaagaagggccaacgctgggggtccctggaacgacgggggatgcaagctatggagggggaggtgttactcccagctctctatgaggaggaagag

SEQ ID NO: 60 - DNA, 255 bp, Homo Sapiens
gaatcgtcgacccacgcgtccgcgcaagcaggaggcgggggagtcggaggaggtggcggcgctggagctcctcccggggaccagcgacccggggagcgagcacgtcgctccgcaccgctcttcctccagccgctgagccgtcccttctcgccatgtcccagagcaggcaccgcgccgaggccccgccgctggagcgcgaggacagtgggaccttcagtttggggaagatgataacagctaagccagggaaaacac

SEQ ID NO: 61 - DNA, 255 bp, Homo Sapiens
gaatcgtcgacccacgcgtccgagaaacctgcagccgcagcagctcctggcactgcggagaagttgagccccaaggcggccacgcttgccgagcgcagcgccggcctggccttcagcttgtaccaggccatggccaaggaccaggcagtggagaacatcctggtgtcacccgtggtggtggcctcgtcgctggggctcgtgtcgctgggcggcaaggcgaccacggcgtcgcaggccaaggcagtgctgagcgcc

SEQ ID NO: 62 - DNA, 255 bp, Homo Sapiens
gaatcgtcgacccacgcgtccgccccacgccgcggcgcgcccgggctccctgctgatcccagaacaatcaaccatgacgaccgaatctggatcagactcggaatccaagccggaccaggaggccgagccccaggaggcggcgggggcgcaggggcgcgcgggggcgcccgtgccggagccgcccaaggaggagcagcagcaggccctggagcagttcgccgccgctgcagcgcacagcaccccggtgcggaggga

SEQ ID NO: 63 - DNA, 255 bp, Homo Sapiens
gaatcgtcgacccacgcgtccgtgagtctcaagatggacaaccgggatgttgcaggaaaggctaaccggtggtttggggttgctccccctaaatctggaaaaatgaacatgaacatccttcaccaggaagagctcatcgctcagaagaaacgggaaattgaagccaaaatggaacagaaagccaagcagaatcaggtggccagccctcagcccccacatcctggcgaaatcacaaatgcacacaactcttcctgc

SEQ ID NO: 64 - DNA, 255 bp, Homo Sapiens
agaatcgtcgacccacgcgtccgcccacgcgtccgccatgccagatgaccttctgaccacgttggatgacacttgtgatctctttgcccccctagtccaggagactaataagcagcccccccagacggtgaacccaattggccatctcccagaaagtttaacaaaacccgaccaggttgcccccttcaccttcttacaacctcttcccctttccccaggggtcctgtcctccactccagatcattcctttagttt

SEQ ID NO: 65 - DNA, 255 bp, Homo Sapiens
gaatcgtcgacccacgcgttccgattgtgatggaccccaagaaggacgtcctcatcgagttctacgcgccatggtgcgggcactgcaagcagctagagcccgtgtacaacagcctggccaagaagtacaagggccaaaagggcctggtcatcgccaagatggacgccactgccaacgacgtccccagcgaccgctataaggtggagggcttccccaccatctacttcgcccccagtggggacaaaaagaacccag

SEQ ID NO: 66 - DNA, 255 bp, Homo Sapiens
gaatcgtcgacccacgcgtccggccccttggagagagaggctgcctcagccagtggcacaggagactccaaggagctactgacattcctaagagtggaggaggaggaggagccttgctgggccagggaaacaaagtttacattgtcctgtagctttaaaaccacagctgggcagggtgagaagctagatgcccctgcagtttggccctggagccagggcagaggaatgtagggcctgcatggagaagggttctgc

SEQ ID NO: 67 - DNA, 255 bp, Homo Sapiens
gaatcgtcgacccacgcgtccggccagctccaccagcacgtgcagcagctacacctgggcacccggctgcctgtggtggtcgtggccaacaaagctgacctgttgcacatcaaacaggttgaccctcagcttggactgcagctagccagcatgctaggctgctcattctatgaagtgtctgtcagtgaaaattataatgatgtctacagcgccttccacgtcctctgtaaagaggtcagtcacaaacagcagcct

SEQ ID NO: 68 - DNA, 255 bp, Homo Sapiens
gaatcgtcgacccacgcgtccggcctccgccttcggaggctgacgcgcccgggcgccgttccaggcctgtgcagggcggatcggcagccgcctggcggcgatccagggcggtgcggggcctgggcgggagccgggaggcgcggccggcatggaggcgctgctgctgggcgcggggttgctgctgggcgcttacgtgcttgtctactacaacctggtgaaggccccgccgtgcggcggcatgggcaacctgcgggg

SEQ ID NO: 69 - DNA, 255 bp, Homo Sapiens
gaatcgtcgacccacgcgtccgcgggcggcggcggagcgggcggcggagcggcggcagcagcggtgagcgcgagccccggagccccgggcggccggcctcccgcgcccgcgcagccccgcgtctgcgtccggccggagacgccgggccccgcgccgcgccgcgccatgtcggtgaacatggacgagctgcggcaccaggtcatgatcaaccagttcgtgctggccgcgggctgcgcggccgaccaggcgaagcag

SEQ ID NO: 70 - DNA, 255 bp, Homo Sapiens
gaatcgtcgacccacgcgtccggagtgtacaatccgtggccggagcctgatccagattagcatccaggaggacccctggaacctccccaactccatcaagaccctggtggacaacattcagagatatgtggaagatgggaagaaccagctgctcctggccttgctgaagtgcacagacacggagctgcagctgcgcagagacgcgatcttctgccaggccctggtggccgccgtgtgcaccttctccgagcagct

SEQ ID NO: 71 - DNA, 255 bp, Homo Sapiens
gaatcgtcgacccacgcgtccgaccaggcatgcagacaagtgcctgcagtgattgcagcgcctgcgcagcggcccttgttggaagtccaggagtccgcgctaccccgcaaagtccctggccgctgcccgcacaccgaacctctcggggctgagtcgggctgctttctcctcgcgtcccggggcgccctcgggaactcgggagcgcgcggcgagctgaacaatggcctggggacacctggcaggtcgcaggcccca

SEQ ID NO: 72 - DNA, 255 bp, Homo Sapiens
  misc_feature (1)..(1): n is a, c, g, or t
naatcgtcgacccacgcgttccgacnaaaacgaaaangaaaatgggtcccaggccctccccgccagcctccgtagacgggaaggatcaggaagcggggactccgggatgtcagcggcgccgnggcgcgggctccgggcttccctgccctgcaatgtggaagctgctctctangaggggcccgaggctgggcccacggatgcgggcgcgccgggaggatatttancnttgaagagcggaaggnggcggagactcgg

SEQ ID NO: 73 - DNA, 255 bp, Homo Sapiens
gaatcgtcgacccacgcgtccgcgggcgcacgcgcgctcctggagtttcccaaccttccgggtgcgggcggggcatgttacgtcacgtcgtttaatccggaaacggcggcggcggcgacaggaccgaggggccttagttggtgggcaagtcggggatcccagaaagagaagcgtgacccggaagcggaaacgggtgtccgtcccagctccggcctgccagtgagcttctaccatcatggacctattgttcgggcg

SEQ ID NO: 74 - DNA, 255 bp, Homo Sapiens
  misc_feature (162)..(162): n is a, c, g, or t
gaatcgtcgacccacgcgtccgggtttggttgtgagatcgagaataacagaagcagcggagcattctggaaatattactatgatggaaaggactacattgaattcaacaaagaaatcccagcctgggtccccttcgacccagcagcccagataaccaagcanaagtgggaggcagaaccagtctacgtgcagcgggccaaggcttacctggaggaggagtgccctgcgactctgcggaaatacctgaaatacagc

SEQ ID NO: 75 - DNA, 255 bp, Homo Sapiens
gaatcgtcgacccacgcgtccgtgaaggccgaggccaagatggcggtgctgtcaggtgagcgcggcaccggcggcgggtgtggggccgcgcgggtctggggccgtgggagcctcgggtgtcgtggtggcgtcgggggtcggtgccggcgtcgtggccgcggtcctctccgggcttctccgagccggccgctcctcgggctccccgcccggcttgcgatgaacggtcgccgttattgcgtccagagtacagtcggg

SEQ ID NO: 76 - DNA, 255 bp, Homo Sapiens
  misc_feature (172)..(172): n is a, c, g, or t
gaatcgtcgacccacgcgtccgccgagtccgcggcggcgtccagggtcggcagcaaccgcagccgagcccgagcgggtggcggcgccatggcgtgcgcggggctgctcaccgtgtgcctgctccggccgcccgcgccccagccccagccccagaccccgcggcacccccagntcgcgcccgacccggggcccgccggacacacgctcttccaggacgttttccgcanagcanacaagaatggagtacgagagggc

SEQ ID NO: 77 - DNA, 255 bp, Homo Sapiens
gaatcgtcgacccacgcgtccgtccacatggagtacagctgcggcacagcggccatccggggcaccaaggagctgggggagggccagcacttctgggagatcaagatgacctctcccgtctacggcaccgacatgatggtgggcatcgggacgtcggatgtggacctggacaaataccgccacacgttctgcagcctgctgggcagggatgaggacagctggggcctctcctacacgggcctcctccaccacaag

SEQ ID NO: 78 - DNA, 255 bp, Homo Sapiens
gaatcgtcgacccacgcgtccgccaagatgccgcccaaaggaaaaagtggttctggaaaagcggggaaagggggagcagcctctgggagtgacagtgctgacaagaaggctcaaggtcccaaaggtggtggcaatgcagtaaaggtcagacacattctatgtgaaaaacatggcaaaatcatggaagccatggaaaagttaaagtctgggatgagattcaatgaagtggccgcacagtatagtgaagataaagcc

SEQ ID NO: 79 - DNA, 255 bp, Homo Sapiens
  misc_feature (140)..(140): n is a, c, g, or t
tagaatcgtcgacccacgcgctccgcgagtcgatcggccgccgcgctcgagcctccgcccgcaccgagccgcgggacccgggccgtaccggggaggggccgctccgggccgcaacgcgagggcagcgaggggcggcgggnacctgncaccgggcggggccggcggcagcgaccatgatcgctttgttcaacaagctgctggactggttcaaggccctattctggaaggagganatggagctcacgctggtcgggc

SEQ ID NO: 80 - DNA, 255 bp, Homo Sapiens
gaatcgtcgacccacgcgtccggactggcgccacggcagcggtgctgcagagcgacaccatggaccattaccgcaccttccacatgctcgagcggctgctgcatgcgccgcccaagctactgcaccagctcatcttccagattccgccctcccggcaggcactactcatcgagaggtactatgcctttgatgaggcctttgttcgggaggtgctgggcaagaagctgtccaaaggcaccaagaaagacctggatg

SEQ ID NO: 81 - DNA, 255 bp, Homo Sapiens
gaatcgtcgacccacgcgtccggaaaagccatcgagctcaacccagccaacgccgtctatttctgcaacagagccgcagcctacagcaaactcggcaactacgcaggcgcggtgcaggactgtgagcgggccatctgcattgacccggcctacagcaaggcctacggcaggatgggcctggcgctctccagcctcaacaagcacgtggaggccgtggcttactacaagaaggcgctggagctggaccccgacaac

SEQ ID NO: 82 - DNA, 255 bp, Homo Sapiens
gaatcgtcgacccacgcgtccgcagcagagctggaggctgaggagctcgagaaattgcaacaatacaaattcaaagcacgtgaacttgatcccagaatacttgaaggtgggcccatcttgcccaagaaaccacctgtgaaaccacccaccgagcctattggctttgatttggaaattgagaaaagaatccaggagcgagaatcaaagaagaaaacagaggatgaacactttgaatttcattccagaccttgccct

SEQ ID NO: 83 - DNA, 255 bp, Homo Sapiens
gaatcgtcgacccacgcgtccgctggaggcgcgggccgggcggtgcgcactgcgggcgcatccctgccccggcgccgtccgtgcccgcgggacctgacagccgggtcagagggcgaagctgtgctcaggcccgggctggacgcagagccagagctgtccccagaggagcagagggtcctggaaaggaagctgaaaaaggaacggaagaaagaggagaggcagcgtctgcgggaggcaggccttgtggcccagcac

SEQ ID NO: 84 - DNA, 255 bp, Homo Sapiens
gaatcgtcgacccacgcgtccgctcagtaccgtgtgactgtgccgctgatgggggctgtgtccgacctgtgcgaggctctctccaggctgtctggcattgctgcagaaaatatggtggtcgcagatgtgtataatcaccgattccacaaaattttccaaatggatgaaggtttaaaccacatcatgcctcgggatgacattttcgtgtacgaggtctgcagcacttccgtggatggctcggaatgtgtcacgctt

SEQ ID NO: 85 - DNA, 246 bp, Homo Sapiens
  misc_feature (1)..(1): n is a, c, g, or t
naatcgtcnacccncncgctccgccctggcggcggcggcggcgcctgacactcggcgcctcctgccgtgctcangggcgnnatgtccnaggctggaggngccngcccgggaggctgatccnncaagatccgggacangntcctgnncccggggcgnctgacccngnatnctgnngcncttccgcccgngccnccgtatggctccnnctgcgntattgcngcnggggngtcggtctnctgcnantnc

SEQ ID NO: 86 - DNA, 255 bp, Homo Sapiens
gaatcgtcgacccacgcgtccggccatcggcgtgctgaccagcggcggggatgctcaaggtatgaacgctgccgtccgtgccgtggtgcgcatgggtatctacgtgggggccaaggtgtacttcatctacgagggctaccagggcatggtggacggaggctcaaacatcgcagaggccgactgggagagtgtctccagcatcctgcaagtgggcgggacgatcattggcagtgcgcggtgccaggccttccgcac

SEQ ID NO: 87 - DNA, 255 bp, Homo Sapiens
gaatcgtcgacccacgcgtccgaaaaaagagaacgagaaagacaagaactgagaattttagtgggaacaaatttggtgcgcctcagtcagttggaaggtgtaaatgtggaacgttacaaacagattgttttgactggcatattggagcaagttgtaaactgtagggatgctttggctcaagaatatctcatggagtgtattattcaggttttccctgatgaatttcacctccagactttgaatccttttcttcgg

SEQ ID NO: 88 - DNA, 255 bp, Homo Sapiens
gaatcgtcgacccacgcgtccgctccccacagccagaggaggccacagacccctcaggggagttccgcgctggggtctgggctgtgctccctcactaaagggaaggaaaggaagctgggcgtcctccgggccccccaacacacgtcccatttagccctgcacagcggtctccttcccctaagccagcactgctgctccctggagcccgggaaggaggctgcctggctggaggcccgagccgatggcgcctgtgct

SEQ ID NO: 89 - DNA, 255 bp, Homo Sapiens
gaatcgtcgacccacgcgtccggctgcctatttccaccaagcagccaagctcaagggaatcggggagtacgtgaacatccgcacagggatgccctgccacttgcaccccaccagctccctttttggaatgggctacaccccagattacatagtgtatcacgagttggtcatgaccaccaaggagtatatgcagtgtgtgaccgctgtggacggggagtggctggcggagctgggccccatgttctatagcgtgaa

SEQ ID NO: 90 - DNA, 255 bp, Homo Sapiens
gaatcgtcgacccacgcgtccgacatcctgcagcagacactgatggacgaggggctgcggctcgcccgcctcgtctcccacgaccgcgtgggccgcctcagcccctgtgtgcctgcgaagccacctctcgcagagttcgaggaagggctgctggacagatgtcctgccccaggaccccatcccgcgctggtggagggtcgcaggagcagcgtgaaagtggaggctgaggccagccggcagtgagggttggactgg

SEQ ID NO: 91 - DNA, 255 bp, Homo Sapiens
gaatcgtcgacccacgcgtccgattgcccgctccaccctccacatgccctttctaactcctcagttgacctagggtccccaagccttgccagtcacccccggggcctgctgcagaaccgtggcctcaacaatgacagtcctgagcggaggcgcaggcctggccatggcagcctgaccaacatcagccggcacgactccctcaagaagatcgacagccctcccattagaaggtccacgtcatcagggcaatacacg

SEQ ID NO: 92 - DNA, 255 bp, Homo Sapiens
  misc_feature (143)..(143): n is a, c, g, or t
gaatcgtcgacccacgcgtccggccgctgccaccgcaccccgccatggagcggccgtcgctgcgcgccctgctcctcggcgccgctgggctgctgctcctgctcctgcccctctcctcttcctcctcttcggacacctgcggnccctgcgagccggcntcntgcccgcccctgcccccgctgggctgcctgctgggcgagacccgcgacgcgtgcggttgctgccctatgtgcgcccgcggcgagggcgagccgt

SEQ ID NO: 93 - DNA, 255 bp, Homo Sapiens
gaatcgtcgacccacgcgtccgcggacgcgtgggtggggccccgggcggcgttgaccatgacccagcagggcgcggcgctgcagaactacaacaacgagctggtcaagtgcatagaggagctgtgccagaagcgggaggagctgtgccggcagatccaggaggaggaggacgagaagcagcggctgcagaatgaggtgaggcagctgacagagaagctggcccgcgtcaacgagaacctggcacgcaagattgcc

SEQ ID NO: 94 - DNA, 255 bp, Homo Sapiens
gaatcgtcgacccacgcgtccggcctgcagagcaacatgcccaagttttattgtgactactgcgatacatacctcacccatgactctccatctgtgagaaagacacactgcagtggaaggaaacacaaagagaatgtgaaagactattatcagaaatggatggaagagcaggctcagagcctgattgacaaaacaacggctgcatttcaacaaggaaagatacctcctactccattctctgctcctcctcctgca

SEQ ID NO: 95 - DNA, 255 bp, Homo Sapiens
gaatcgtcgacccacgcgtccgcccacgcgtccgctagtccaagtgtccggcaaacttggatccatgaaatcaaccaaattttagaaaaccagcgcaattttttaaatgccttgacatcgccaatcgagtaccagaggaaccacagcgggggcggcggcggcggcggcagcgggggcagcggcgggggtgggggcagcggcggcggcggggcccccagtggcggcagcggccacagtggcggccccagcagctgc

SEQ ID NO: 96 - DNA, 255 bp, Homo Sapiens
gaatcgtcgacccacgcgtccgatgaaattgaagaaatgggtggaatggccaaagctgtagctgagggaatacctaaacttcgaattgaagaatgtgctgcccgaagacaagctagaatagattctggttctgaagtaattgttggagtaaataagtaccagttggaaaaagaagacgctgtagaagttctggcaattgataatacttcagtgcgaaacaggcagattgaaaaacttaagaagatcaaatccagc

SEQ ID NO: 97 - DNA, 255 bp, Homo Sapiens
gaatcgtcgacccacgcgtccgcggaccctcgacgctgcatgaggcaccactatgtggattctatcagtcacccattgtacaagtgtagctcaaagatggtgctcctggccaggtgcgaggggcactgcagccaggcgtcacgctccgagcctttggtgtcgttcagcactgtcctcaagcaacccttccgttcctcctgtcactgctgccggccccagacttccaagctgaaggcactgcggctgcgatgctca

SEQ ID NO: 98 - DNA, 255 bp, Homo Sapiens
  misc_feature (1)..(1): n is a, c, g, or t
naatcgtcgacccacgcggtccgggacgccgacggcggcgggcgcttccgcctcgcgggcctcggcccggtgcgagcggctccgcgatgtggctgagcccggaggaggtgctggtggccaatgcgctgtgggtgacggagcgggccaaccccttcttcgtgctgcagcgacgccggggccacggcaggggcggcggccttacgggtcttctcgtgggcaccctggacgtggtgctggactccagtgcccgcgtgg

SEQ ID NO: 99 - DNA, 255 bp, Homo Sapiens
  misc_feature (34)..(34): n is a, c, g, or t
gaatcgtcgacccacgcgttccgcccacgcgtcnggtacgcntccccacgaggggctggttcctgaaaggatgtacagaggatccccaaccgcctgcgaaacccaagccgccgcgtaggagcgtgcgttcgggccctcttctcccacctgttcgactccccatccccaggatgtcaacctcagtccctcaaggccatacctggacccaacgggtgaagaaagacgatgaggaggaggacccgctggaccagctga

SEQ ID NO: 100 - DNA, 255 bp, Homo Sapiens
gaatcgtcgacccacgcgtccgcccacgcgtccggcagagcttccgggctgcgctcttcgttgcccagtttccgctcagtggtcgcgtctccgccccccacccaccagtcccgctgcattctcggccgggctctaggcgccatggctccccgcgggaggaagcgtaaggctgaggccgcggtggtcgccgtagccgagaagcgagagaagctggcgaacggcggggagggaatggaggaggcgaccgttgttatc

SEQ ID NO: 101 - DNA, 255 bp, Homo Sapiens
gaatcgtcgacccacgcgtccgcccacgcgtccgcaggtgcaatcgtggaataccacgggcctggtgtagactccatctcctgcactggcatggcgacaatctgcaacatgggtgcagaaattggggccaccacttccgtgttcccttacaaccacaggatgaagaagtacctgagcaagaccggccgggaagacattgccaatctagctgatgaattcaaggatcacttggtgcctgaccctggctgccattat

SEQ ID NO: 102 - DNA, 255 bp, Homo Sapiens
gaatcgtcgacccacgcgtccggccgggcgcctcttgcaatggagacggtcatttcttcagatagctccccagctgtggaaaatgagcatcctcaagagaccccagaatccaacaatagcgtgtatacttccttcatgaagtctcatcgctgctatgacctgattcccacaagctccaaattggttgtatttgatacgtccctgcaggtgaagaaagctttttttgctttggtgactaacggtgtacgagctgcc

SEQ ID NO: 103 - DNA, 255 bp, Homo Sapiens
gaatcgtcgacccacgcgtccgaggcagtcagcgaggcccaggagaaattgacagaatccaaccagaagctggggctgctgcgggaggctctggagcggagacttggggagctgcccgccgaccaccccaaggggcggctgctgcgagaagagctcgctgcggcctcctccgctgccttcagcacccgcctggccgggccctttcccgccacgcactacagcaccctgtgcaagcccgcgccgctcacagggacc

SEQ ID NO: 104 - DNA, 255 bp, Homo Sapiens
gaatcgtcgacccacgcgtccgcccacgcgtccgattctggagaagccggtggacaagcaggacgcctacaggatgctgtcccggttgagtgggagagaacacagcgtgttcacaggtgtcgcgatcgtccactgctccagcaaagaccatcagctggacaccagggtctcggaattctacgaggaaacgaaggtgaagttctcggagctgtccgaggagctgctctgggaatacgtccacagcggggagcccat

SEQ ID NO: 105 - DNA, 255 bp, Homo sapiens
gaatcgtcgacccacgcgtccgccgagctgggatcgggccccgggcgggggcggtgcgagcggcgccaagcagatcttaggggcggggacggagccggggcgggcgggactgaagcggagcccgggaacggggcgggaggtcccagggtcccgggttgggggggtggagcagcatttcgtcgccgcgggggtgccgggactccggccgcagtgtcgccgccatcacggacttcctgtgggacaagcgcacgggcc

SEQ ID NO: 106 - DNA, 255 bp, Homo Sapiens
gaatcgtcgacccacgcgtccgggaatcctatcaaaattggttatggtaaagctacacccaccacccgcctctgggtgggaggcctgggaccttgggttcctcttgctgccctggcacgagaatttgatcgatttggcaccatacgcaccatagactaccgaaaaggtgatagttgggcatatatccagtatgaaagcctggatgcagcgcatgctgcctggacccatatgcggggcttcccacttggtggccca

SEQ ID NO: 107 - DNA, 255 bp, Homo Sapiens
agaatcgtcgacccacgcgtccgcccacgcgtccgcggacgcgtgggctccacggccgaagaatgtgatctttgaagatgaggagaaatccaagatgctggcccgcctgctgaagagctcccatcccgaagacctccgcgcagccaataagctcatcaaagagatggtgcaggaggaccagaagcggatggagaagatctcgaagagggtgaatgccatcgaggaggtgaacaacaatgtgaaactgctcacgga

SEQ ID NO: 108 - DNA, 255 bp, Homo Sapiens
  misc_feature (9)..(9): n is a, c, g, or t
gaatcgtcnacccncgcgtccgggaagaactggagaaaaggaaggatganattgaacgagaanttctccgaagggtggaggaagccaaacgcatcatggaaaagcagttgctcgaagaactcgagcgacagagacaagctgagcttgccgcacaaaaagctagagagaggaagctggcgcgtatggcagccgaggagcacactctgcaggacactggacaagacagaggaagaacgtgcaaaacgtgaggagcta

SEQ ID NO: 109 - DNA, 255 bp, Homo Sapiens
gaatcgtcgacccacgcgtccgccatccgggatgctaacctgtacgtcagcgggctccccaagaccatgagccagaaagagatggagcagctcttctcccagtacggccgcatcatcacgtcccgcatcctggtggaccaggtcacaggtgtctctcggggtgtgggattcatccgctttgacaagaggattgaggccgaagaggctatcaaaggactgaatgggcagaagccgctgggcgcagctgagcccatc

SEQ ID NO: 110 - DNA, 255 bp, Homo Sapiens
  misc_feature (31)..(31): n is a, c, g, or t
gaatcgtcgacccacgcgtccggggaggggngcccgaggncgttgcngacctnggngctanagccgctgctgcctnggaagacagatcctccagtacncgcctncngtcngngcagagtgncgtgtcctcctcccacccctagcngnncnccntcncgganngatttcntaaacggccttnngncactagtcgatccgagcngccgccatccgtnactgcntgggttgntgacactccngttggcaaggtccact

SEQ ID NO: 111 - DNA, 255 bp, Homo Sapiens
  misc_feature (111)..(111): n is a, c, g, or t
gaatcgtcgacccacgcgtccgccttgagggaagaagaaaggagagaatctgctgcagcagacgctggctttgctctctttgagaagaagggagccttagctgaaaggcancagctcatcccanaattggtgcgaaatggagatgaggagaatcccctcnaaaggggtctggttgctgcttacagtggtgacagtgacaatgaggaggagctggtggagagacttgagagtgaggaagagaagctagctgactgg

SEQ ID NO: 112 - DNA, 255 bp, Homo Sapiens
gaatcgtcgacccacgcgtccgtgaaaccacagcctacgccctgctgcacctcctgcttcacgagggcaaagcagagatggcagaccaggctgcggcctggctcacccgtcagggcagcttccaagggggattccgcagtacccaagacacggtgattgccctggatgccctgtctgcctactggattgcctcccacaccactgaggagaggggtctcaatgtgactctcagctccacaggccggaatgggttca

SEQ ID NO: 113 - DNA, 255 bp, Homo Sapiens
gaatcgtcgacccacgcgtccggcgtgtttcactgctgcaccttctgctccagcgggggccagaaaaccgctcgctgcgcctgtggaggcaccatgccaggtgggtacctaggctgtggccatggacacaaaggccacccaggtcatccccactggtcatgctgtggaaaatttaatgagaaatctgaatgcacatggacaggtgggcagagcgcaccgaggagtctacttaggactgtggctctctgatgggtt

SEQ ID NO: 114 - DNA, 255 bp, Homo Sapiens
gaatcgtcgacccacgcgtccgaggacccccaccttcgctcctgggagccccgctgcaccccaggagttccattccagcctccccagtgcgagtgcggccgagcgctgacttttgggggctccctcccccgtctcactgggtccctctgcggctcccccgcccgctgtcccggttcagacgtggctgcaccccggaggggggcgccggccgagacctgagacgtggcgctccctctctgccggcctcggcttcag

SEQ ID NO: 115 - DNA, 255 bp, Homo Sapiens
gaatcgtcgacccacgcgtccgacgccatcctggtgctggcgcccaaaggctccctgggggaccgcttctgccgcgaccgcctgcccctcctggacattgcttgcaacaagttcctgacctgcagcgtggaggatggggagctggtcttccgccacgcccaggacctcatcctggagatcatctacactgagcccgtcgacctgtccctgggcaccctgggggagatcagtgggcaccagctcatgagtctgtct

SEQ ID NO: 116 - DNA, 255 bp, Homo Sapiens
gaatcgtcgacccacgcgtccgggcatcacgaaacattggatcatgacatgtcgggcgatgcttggaagagcccagcatgtatgtatgcacacattgtgtgtgtgggaaggacaaagccactctcacaagaaagggcaccaggactgctctccaaggaactggacctgtccagacagttacactccaaggtcattggagagaacttctgtatgggcaagcctgagagggagaggaaacaaaagctgtgtcctggc

SEQ ID NO: 117 - DNA, 255 bp, Homo Sapiens
gaatcgtcgacccacgcgtccggagctttccttctttgtcgtttttcttgtggatttatctttctttttatgtttggcagcttgatgatcacatttagtttgactcctttgtacccacactgggagaagcaaagaagaacacttggccttaaaggatgggcaaggctgggcacagtggcccacacctggatcccagccttagaggatgggcgaggccgggcacagtggcccacacctggatcccagccttagagg

SEQ ID NO: 118 - DNA, 255 bp, Homo Sapiens
gaatcgtcgacccacgcgtccgcaggagaggctcagcctagactatacagtgactccggggagcaccagaaaagaccctgcccaggccccacccagaccacaacatccacctctctggtggggccagtgtgtggtgggggcctcccgttagaggtagatgtgtgcatgcctggtgcagggggctggtgcagagcattggcaggagtggcacaggaggccggcggcacctggagctccgcagcatccacccaggcc

SEQ ID NO: 119 - DNA, 255 bp, Homo Sapiens
gaatcgtcgacccacgcgtccgcggagatcctgcgctccggcaagcggccagacctagtgtcctacgtgcagactctgtgcaagggtctgtcgcagcccaccaccaatctggtggccggctgtctgcagctcaactctcgcaacttcctcacggagcaaggcgccgacggtgccggccgcttccacggctcgggcggcccgttcgccatgcacccctacccgtacccgtgctcgcgcctggcgggcgcacagtgc

SEQ ID NO: 120 - DNA, 255 bp, Homo Sapiens
gaatcgtcgacccacgcgtccgcccacgcgtccgctccagaccacagaaggggagtcctggggacaggactggtgtagacaggcatctccaccgccccggttagccagtacctaacacccactcctgccctcggccgatgtggcaaaccggggaggcttttgtgtggggccggggctgcggccatggcagccaccctccagctcccgggggctggggaagacgctggggtccccgtggaggctggagcggcgttc

SEQ ID NO: 121 - DNA, 255 bp, Homo Sapiens
gaatcgtcgacccacgcgtccgggtctacaccgacttcgacggcacccgagtgtacagccccccggagtggatccgctaccaccgctaccacgggcgctcggccaccgtgtggtcgctgggcgtgcttctctacgatatggtgtgtggggacatccccttcgagcaggacgaggagatcctccgaggccgcctgctcttccggaggagggtctctccagagtgccagcagctgatccggtggtgcctgtccctgc

SEQ ID NO: 122 - DNA, 255 bp, Homo Sapiens
  misc_feature (39)..(39): n is a, c, g, or t
gaatcgtcgacccacgcgtccgcccacgcgtccgcccangcgtccggagccatggntcagaaaatggactgtggtgcgggcctcctcggcttccaggctgaggcctccgtanaagacagcgccttgcttatgcanaccttgatggaggccatccagatctcanaggctccacctactaaccaggccaccgcagctgctagtccccagagttcacagcccccaactgccaatgagatggctgacattcaggtttca

SEQ ID NO: 123 - DNA, 255 bp, Homo Sapiens
gaatcgtcgacccacgcgtccggaagccctgaaggagcaggaggagatcaacttccggctgaggcagtacatggacaagattatcctcgccatcctggaccacaatccctccatcctcgagatcaaacactaaggcacggggctggctgcagagcagccttaggaccctgggaccaagggcagaccctgcccaaggatgcaggcctaagccgggcctcacactcacactgtaaatgtctctctggccaccatgcg

SEQ ID NO: 124 - DNA, 255 bp, Homo Sapiens
gaatcgtcgacccacgcgtccggtcaggtagactgtgaggagaaagtgaagggttttaagtggggaaatgacatgatttggtttttattttaggattacaggcgtgagccgccgcgcctggctgcttgcagcctttatattatctatggctgctattatataccctctccagttctgctgcagtggcataatagagtaattgtgccgagaatgaatttgtctctaggcccaaaagcctaaaatatctacattctg

SEQ ID NO: 125 - DNA, 255 bp, Homo Sapiens
taatcgtcgacccacgcgttccgaccagcagaatgccgcgtgcgagtggctgctgggggaccggaagccctctccggaggagctggacaagggcatcgaccccgacagtcctctctttcaggccatcctggataacccggtggtgcagctgggcctgaccaacccgaaaacattgctagcatttgaagacatgctggagaacccactgaacagcacccagtggatgaatgatccagaaacggggcctgtcatgct

SEQ ID NO: 126 - DNA, 255 bp, Homo Sapiens
gaatcgtcgacccacgcgtccgccagcccccgcgcccgcccgccagcccgcccagcggtcgggtccgggcgcccgcgcagaatcagctgtctgagctgcccaggcggcgggggagcagcgagcgggcttcagcgagccgcaggaggcacaggcctgtcctgggtccccgcaggtctgcgcgtctgttgttcccagcgctctgagaggcctgaaaaggaagagcaacctgtccagaatccccgcaggaaaggaaaa

SEQ ID NO: 127 - DNA, 255 bp, Homo Sapiens
gaatcgtcgacccacgcgtccggacgccatgacgccccggctggtgtgtgtcggtgtgtatgtgtgtgtgtgagtgtgcgcgctccgagtgtgtgtgtatttgtgtatcggcggtcccgcaggtcccggatgttgcggacagtatgaggcaagcgcagggggcggggaccagcagctgtcgccgccgctctcaggctctgggaaccacccttctactttctgtctctaggaatttcactactctagggtgaagag

SEQ ID NO: 128 - DNA, 255 bp, Homo Sapiens
gaatcgtcgacccacgcgtccggtcggtgtgtatgtgtgtgtgtgagtgtgcgcgctccgagtgtgtgtgtatttgtgtatcggcggtcccgcaggtcccggatgttgcggacagtatgaggcaagcgcagggggacggggaccagcagctgtcgccgccgctctcagggtgaagagggaacagaaatctttgccccctgactttggaaatctcgtttaaccttcaaactggcgatgtcaagggttccaagtcct

SEQ ID NO: 129 - DNA, 255 bp, Homo Sapiens
gaatcgtcgacccacgcgtccggtgggagtgcgggtgggggtgtggccgagccccggcaggaagccccgcccagacggtgttcagggaacccggagcccaagcgctccggcggagcccaaaagggtgggggtgggaggggcagaggccaacggatccccctgcctgtcgcaccccttggcgggagacgggaaggcagcgggctgcgtacgatgggaccctggtgcagacgccgggccggctgacatttggacccc

SEQ ID NO: 130 - DNA, 255 bp, Homo Sapiens
gaatcgtcgacccacgcgtccgcgggagggcagggccgacggacggcgggcggcggcggcggtggcggcgctggagtcggcgcgggtgctggcgccatggaggccgagcggggtcccgagcgccggcctgcggagcgtagcagcccgggccagacgccggaggagggcgcgcaggccttggccgagttcgcggcgctgcacggcccggcgctgcgcgcttcgggggtccccgaacgttactggggccgcctcctg

SEQ ID NO: 131 - DNA, 255 bp, Homo Sapiens
agaatcgtcgacccacgcgtccgttgagagcttgggctcctccgtgagccgccggcgcgcttcgctgcctggcggcctttccccgagccgagcccgcttcccaaggccgccagtcgtcgcctgcccgcgcctgcgcagcgtcccgggccgagagggccacggcggcggccatggcgcaccgctgtttgcggctgtggggccggggcggctgctggccccgcggcctacagcagctcctcgtgcctggcggcgtgg

SEQ ID NO: 132 - DNA, 255 bp, Homo Sapiens
gaatcgtcgacccacgcgtccgccggaccgggacgcagagtctgcggacccggcgccgaggcggccacccgagacgcggcgcgcacgctccggcctgcgcccggcccggccatggcggccccccgcccgtctcccgcgatctccgtttcggtctcggctccggctttttacgccccgcagaagaagttcggccctgtggtggccccaaagcccaaagtgaatcccttccggcccggggacagcgagcctcccccg

SEQ ID NO: 133 - DNA, 255 bp, Homo Sapiens
gaatcgtcgacccacgcgtccgccgaagggccgggacctggctcaggggacgaagccaatgggccccggggagagaggcagacccggactcggggcccggccccatctgcaatgccccagagcaggtctaccgagtcagcccatgctgccaccctgccgcctcgaggcccagagccatctgcccaggagcagatggaggggatgctgtgccgcaagcaggagatggaggccttcgggaagaaggctgccaacagg

SEQ ID NO: 134 - DNA, 255 bp, Homo Sapiens
gaatcgtcgacccacgcgtccggcaacccaggcagccacggctgtttcggagctcaggactctaaaatggcagagcagctttctccaggaaaggcggtggatcaggtgtgcaccttccttttcaaaaagcctgggcggaaaggggctgctggacgcagaaagcgcccggcctgcgacccagagcccggagaaagcggcagcagtagcgacgaaggctgcactgtggttcgaccggaaaagaagcgggtgacccac

SEQ ID NO: 135 - DNA, 255 bp, Homo Sapiens
gaatcgtcgacccacgcgtccgggccttgccccgctctgcagcacagacaggccagatgcatttgtcctttgcctagctactccccaggtagagagtgctcctggtggcctggcaggtctgggcccttctctccctgcccaggttgtccctggagggcagccctcactccctttgggggagaggcagacattgctgcccacagacctgcctctgactcaactgtgtccaccctccctggtccctacccccaagtc

SEQ ID NO: 136 - DNA, 255 bp, Homo Sapiens
gaatcgtcgacccacgcgtccgagttcaagcagctggctctggaggacgccaaagaaggctacagatatggtttggagtgcctttttcgatactacagttatggcctggaaaagaagttccggctggacatattcaaggattttcaggaggaaacggtgaaggactatgaagctggccaactgtatgggctggagaagttctgggccttcttgaaatattccaaagccaaaaatttggacattgaccccaaactg

SEQ ID NO: 137 - DNA, 255 bp, Homo Sapiens
gaatcgtcgacccacgcgtccgctcctccagtgctgtgcgcatggcagtgggctgcctgctggagctggccttcaaggtggctgcaggagagctcaagaatggatttgccatcatccggcccccaggacaccacgccgaggaatccacagccatgggattctgcttcttcaactctgtagccatcaccgcaaaactcctacagcagaagttgaacgtgggcaaggtcctcatcgtggactgggacattcaccatg

SEQ ID NO: 138 - DNA, 255 bp, Homo Sapiens
gaatcgtcgacccacgcgtccggcaggtcagcagattcctggctgcagcttggagggccccagactttgtgcctcgttactgtaaactctatgagcacttgcagagagcaggctccgagctgtttgggcctcgggcagccttcatgctggctctgcgcagtggcttctctggcgccttgctgcagcagtccttcctcactgctgctcacatgagtgagcagtttgccaggtacattgaccaacagatccagggtg

SEQ ID NO: 139 - DNA, 255 bp, Homo Sapiens
  misc_feature (61)..(61): n is a, c, g, or t
gaatcgtcgacccacgcgtccgcacagagctcagtgcaaccaccagaaggagacagtgaancaaaagctcctgaagaatcatcagaggatgtgacaaaatatcaggaaggagtatctgcagaaaacccagttgagaaccatatcaatataacacaatcagataagttcacagccaagccattggattccaactcaggagaaagaaatgacctcaatcttgatcgctcttgtggggttccagaagaatctgcttca

SEQ ID NO: 140 - DNA, 255 bp, Homo Sapiens
gaatcgtcgacccacgcgtccgcggaagcgggggtgcagcgcggcagaatgagggttgattcctcggctgaccccacaatgtcgcaggagcaagggccggggtcctccacgcctcccagttctccgacacttcttgacgctctgctccagaacctttacgactttggaggtacagaaggtgaaacagaacagaagaagatcataaagaaaagggaaaacaagaagagagatgtgatggcttcagcggccttggca

SEQ ID NO: 141 - DNA, 255 bp, Homo Sapiens
gaatcgtcgacccacgcgtccgaccgcggggaggctcacctcgagctgaacgccttccgcaggaagcatgattgtgccctggtcatctcgggagacttcctggaggtttgcctcaagtactatgagtacgagttcatggagctggcctgccagtgcccggccgtagtctgctgccgatgtgcccccacccagaaggcccagatcgtgcgcctgcttcaggagcgcacgggcaagctcacctgtgcactcagtaaa

SEQ ID NO: 142 - DNA, 255 bp, Homo Sapiens
gaatcgtcgacccacgcgtccgcgagaggccagctcagacctcccggctcgacaggcggcgcgggcggcggtaaaatgtcggttccaggaccttaccaggcggccactgggccttcctcagcaccatccgcacctccatcctatgaagagacagtggctgttaacagttattaccccacacctccagctcccatgcctgggccaactacggggcttgtgacggggcctgatgggaagggcatgaatcctccttcg

SEQ ID NO: 143 - DNA, 255 bp, Homo Sapiens
  misc_feature (103)..(103): n is a, c, g, or t
gaatcgtcgacccacgcgtccgataagcccaggcccaacttgggatatcaaacgggaacacgacgttaggaaaccaaaggagctttcagcctgcggccagaanaagcagcgcctcggggagcagagggagcgctcggcgagtccgcagagggccgcgcggccgaggctcgaggaggcgcccggcggccatgggcggcccgaggccggcaggcccgcctcggaggccagggcgcccgggctcgnggcggcagacgc

SEQ ID NO: 144 - DNA, 255 bp, Homo Sapiens
gaatcgtcgacccacgcgtccgataaatccctggggccagtgtcattcaaggacgtggctgtggacttcacccaggaggaatggcagcagctggatcctgagcagaagataacttacagggatgtgatgctggagaactacagcaatctagtttctgtggggtatcacattatcaaaccggatgttatcagcaagttggagcaaggagaagagccatggatagtagaaggagaattcctacttcagagctatcca

SEQ ID NO: 145 - DNA, 255 bp, Homo Sapiens
gaatcgtcgacccacgcgtccgagccgccatcttgtgggagcaaaaccaacgcctggctcggagcagcagcctctgaggtgtccctggccagtgtccttccacctgtccacaagcatggggaacatcttcgccaacctcttcaagggcctttttggcaaaaaagaaatgcgcatcctcatggtgggcctggatgctgcagggaagaccacgatcctctacaagcttaagctgggtgagatcgtgaccaccattcc

SEQ ID NO: 146 - DNA, 255 bp, Homo Sapiens
gaatcgtcgacccacgcgtccgccagcttcctgccagaccgcgaccgcgaggaggaggagaaccggctccgagaggagctgcgccaagagtgggaggcgcagcgcgagaaagtgaaggacgaggagatggaggtcaccttcagctactgggacggctcgggccaccggcgcacggtgcgggtgcgcaagggcaacacggtgcagcagttcctgaagaaggcgctgcaggggctgcgcaaggacttcctggagctg

SEQ ID NO: 147 - DNA, 255 bp, Homo Sapiens
gaatcgtcgacccacgcgtccgaattgattccagacagcattggaaaagacatagaaaaggcttgccaatctatttatcctctccatgatgtcttcgttagaaaagtaaaaatgctgaagaagcccaagtttgaattgggaaagctcatggagcttcatggtgaaggcagtagttctggaaaagccactggggacgagacaggtgctaaagttgaacgagctgatggatatgaaccaccagtccaagaatctgtt

SEQ ID NO: 148 - DNA, 255 bp, Homo Sapiens
gaatcgtcgacccacgcgtccgcccacgcgtccgcccacgcgtccgcccacgcgtccgataagacggcgtcggtgttgcagtctgtgtccttggaggtgaccagggccactgcaggcatggtgctagcagagctgtacgtctctgaccgagagggaagcgatgccacgggagatggaaccaaggagaaaccatttaaaacaggtctaaaggctttgatgacagtagggaaagaaccatttcctaccatttacgta

SEQ ID NO: 149 - DNA, 255 bp, Homo Sapiens
gaatcgtcgacccacgcgtccggtgaaggcctcgttgagagaaggtctcattcggtgttttgggaagagagtcgtgtgggcccaggtctgtctgctatcagctatgccgctgcccgttgcgctgcagacccgcttggccaagagaggcatcctcaaacatctggagcctgaaccagaggaagagatcattgccgaggactatgacgatgatcctgtggactacgaggccaccaggttggagggcctaccaccaag

SEQ ID NO: 150 - DNA, 255 bp, Homo Sapiens
gaatcgtcgacccacgcgtccgctgagatgagcaacctggacctaaccaagattctgtccaagaaatacaaggagcttccggagaagaagaagatgaaatatattcaggacttccagagagagaaacaggagttcgagcgaaacctggcccgattcagggaggatcaccccgacctaatccagaatgccaagaaatcggacatcccagagaagcccaaaaccccccagcagctgtggtacacccacgagaagaag

SEQ ID NO: 151 - DNA, 255 bp, Homo Sapiens
gaatcgtcgacccacgcgtccgcggacgcgtggggtcgctgccgctgccgctgccactgccactgccacctcgcggatcaggagccagcgttgttcgcccgacgcctcgctgccggtgggaggaagcgagagggaagccgcttgcgggtttgtcgccgctgctcgcccaccgcctggaagagccgagccccggcccagtcggtcgcttgccaccgctcgtagccgttacccgcgggccgccacagccgccggccg

SEQ ID NO: 152 - DNA, 255 bp, Homo Sapiens
gaatcgtcgacccacgcgtccgctggagatccaaagggatttgcgtttgtggaatttgaaacaaaagaacaagcagcaaaagcaattgagtttcttaacaacccaccagaagaagcaccaagaaaacctggcatatttcctaaaacagtgaaaaataagcccattccagccttaagagttgtggaagagaagaaaaagaaaaagaagaagaaaggccgaatgaaaaaggaagacaatatccaagccaaagaagaa

SEQ ID NO: 153 - DNA, 255 bp, Homo Sapiens
gaatcgtcgacccacgcgtccgggccgttgtcaccatgccggtcgtccggaagattttccgtcgccgccggggcgactcggagtcagaggaagatgagcaggactcagaggaggttcgattaaaactggaagagaccagagaagtacagaacttgaggaagaggcccaacggggtgagtgctgtggccttgctggtgggagagaaggtacaagaggagaccactctagtggatgatccctttcagatgaagacag

SEQ ID NO: 154 - DNA, 255 bp, Homo Sapiens
gaatcgtcgacccacgcgtccggccgctgtgccccgcgccgcatttctctccccgctgcttccccttctcctgggcttcctgctcctctccgctccgcatggcggcagcggcctgcacaccaagggcgcccttcccctggatacggtcactttctacaaggtcattcccaaaagcaagttcgtcttggtgaagttcgacacccagtacccctacggtgagaagcaggatgagttcaagcgtcttgctgaaaactc

SEQ ID NO: 155 - DNA, 255 bp, Homo Sapiens
gaatcgtcgacccacgcgtccgagaaagtcatgtataagggactcgtccccgaggataaaacattgagagaaataaaagtgaccagtggggccaagatcatggtggttggctccaccatcaatgatgttttagcagtaaacacacccaaagatgctgcgcagcaggatgcaaaggccgaagagaacaagaaggagcctctctgcaggcagaaacaacacaggaaagtgttggataaaggaaaacctgaagatgtg

SEQ ID NO: 156 - DNA, 255 bp, Homo Sapiens
gaatcgtcgacccacgcgtccgcccacgcgtccgcgctgacgggcacgctgactggaggctggcggacaggcgacagcgacctgcggcaggaccagaggagcgagagcagcaagaaccacacccagcagcaatgtcagcggaagtggaaacctcagagggggtagacgagtcagaaaaaaagaactctggggccctagaaaaggagaaccaaatgagaatggctgacctctcggagctcctgaaggaagggacca

SEQ ID NO: 157 - DNA, 255 bp, Homo Sapiens
gaatcgtcgacccacgcgtccgccttcaggagccctgagcccaacatccatgagacaccccgagttgtgcttcatgagagttggattctctgcccactctttcagggatgtccaaggtccatgttagggggcaggcccccagctccactcactgcacaagcaccctaaatgttggaagcacaaaatggttgtgatttgagcacaatggcagggtcccgcaggcagacttgacaggcccaggcccgggaggatccc

SEQ ID NO: 158 - DNA, 255 bp, Homo Sapiens
gaatcgtcgacccacgcgtccggccgaagcggctgcatctggcgccgcgtctgccccgcgtgctcggagcggattctgcccgccgtccccggagccctcggcgccccgctgagcccgcgatcacttcctccctgtgaccaaccggcgctgcaggttagagcctggcaatgccgtttgggtgtgtgactctgggcgacaagaagaactataaccagccatcggaggtgactgacagatatgatttgggacaggtca

SEQ ID NO: 159 - DNA, 255 bp, Homo Sapiens
gaatcgtcgacccacgcgtccggcacagtctccagagatgaagtgagaaggaaaaaagcagtttataagaaggctgaacttgctaaaaaaacagaagttcaggcccactctccctccaggaaattcattttaaaacctgctatcaaatatactagaccaactcatctctcctgtgttaagcggaaaaccacagcagcaggtggggaatcagctctggctcccagtgtatttaaacaggcaaaggacaaagtctct

SEQ ID NO: 160 - DNA, 255 bp, Homo Sapiens
gaatcgtcgacccacgcgtccgcccacgcgtccggagagcgcctgcctgtgcgccccgagcggggctgggactcttccaagatgcccacgttcgcacagagaccccggatcgcggaagctcgcgtctcgaaaggcctgcggtctcacgccctgcccgtcctgggttcacggtttttcatcacctgcggctgtcctgcgatcgaccacagctgtgcaggaggggcaggaggtatctgttgctgcagttaccggaac

SEQ ID NO: 161 - DNA, 255 bp, Homo Sapiens
gaatcgtcgacccacgcgtccgctacccttaaaggacaaagatttaccaacactgaagacccttacaagttgacagtttctaaagagggttctaggaatgttctgtgtaaggtcagcttcactggaataaacatacatcttcccaaggtgactacttcaaaggcaacactacccattcgggtgcagaaattaccgtacttactctgccataaatgtaaaatgtgtataaagttaagctattgcattttttcataa

SEQ ID NO: 162 - DNA, 255 bp, Homo Sapiens
gaatcgtcgacccacgcgtccgatctgccccatctgcttcaagaacgagatggacttctggaaggtggacctcaagttcctggacgactgttgcaagagccacctgagcgagaagcgcgaggagctggaggagatcgcgcgccgcgtgcagctcatcctggacgacctgggcgtggacgcggccgagggccgctggcgccgctgccagaagtgcgtctggaagttcctggagaagcccgagtcgtcgtgcccggc

SEQ ID NO: 163 - DNA, 255 bp, Homo Sapiens
  misc_feature (51)..(51): n is a, c, g, or t
gaatcgtcgacccacgcgtccgcgattggtgtcctgtcaccatggcgctgnctgtcttgcgggtcctggagccctttccgaccgagacacccccgttggcagtgctgctgccacccgggggcccgtggccggcggcggagctgggcctggtgctggccctgaggcctgcaggnnanagcccngcagggccggcgctgctggtggcagccctggaggggccggacgcgggcaccgaanagcagggtcccgggccgc

SEQ ID NO: 164 - DNA, 255 bp, Homo Sapiens
gaatcgtcgacctgcaagatctgcggccgctccctatagtgagtcgtattaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaat

SEQ ID NO: 165 - DNA, 255 bp, Homo Sapiens
gaatcgtcgacccacgcgtccgggtagactcaaagaggtgtgaggtcagagcagacatttgagggggcagcatccctttgggacctcggcccctgctcccctttacccactggtggaggcgggaggttgggtgagagttgggtgtatgttttctgccctgtccacctgtgcaccaggccagggacaccttcaacaatgcctgctgtgccagtgtttgtgtgggtcactgaccctgtgtaccagactcacttggta

SEQ ID NO: 166 - DNA, 255 bp, Homo Sapiens
gaatcgtcgacccacgcgtccggcggccgtacccatggtggaggcctttgctcttcttgtggcgtgctggggtgggcaggtgggctgcagccccagcaccagcctgctccagctcccacaccgcccacagacctccctgtcctcatattctctgaccgcaggcctgcctgcctctgctctgctccgccccagaggggctgggttggacctggtgttcaaggccattcctatctgccccccagcccactgctggag

SEQ ID NO: 167 - DNA, 255 bp, Homo Sapiens
gaatcgtcgacccacgcgtccgggccagccccttgtcctctgccttctgctggcagaggagcagctggactggggccttttggcacagcagccggtgtctcctgcgcccgcctcccccatggccccatgcagccccaggggcttcccccctgcccatggagtagagcccgagatcctggccactatgccagttctgacctcgcatccccctaccccgagcccatgcagtctgggaacatgccgccttctctccag

SEQ ID NO: 168 - DNA, 244 bp, Homo Sapiens
  misc_feature (1)..(1): n is a, c, g, or t
naatcgtcgacccacgcgntccggccagtccgnccgtccggannccggctcgctgggncagcatggcggngtcnccgctgctctgggggccgcggntcgggngcatccgccttttggngctgcgtgctgctcggcctgtttcggccnacccccgngctctgngcgcggncggtaaatganccncgcngcntaaacgnaagngtctccnccngttggctganantggngcncntcnccnnttcnt

SEQ ID NO: 169 - DNA, 255 bp, Homo Sapiens
gaatcgtcgacccacgcgtccggtgagaaccgagccacagtcatgtttcctacatcagcctcctgctcaggggtttgtacagagaccactgccagccctctaggtggtccctgcaaagagagtcccagcccccaaggctccaaatggacagggcaccatgttgaccccttcgtcggcctccagttcgccgactgtaacatgtttcatccagttcaggacgtttatttatttccaacttgcatacaaaacatactg

SEQ ID NO: 170 - DNA, 255 bp, Homo Sapiens
gaatcgtcgacccacgcgtccgcccacgcgatccgaagagatcgagctccctggctgccggctcgccttctgcgtggagttctcgcggtctgggtttcgctgtctgctcttggcccggggtcattttgtcggcgtcgggtgccctctcttgcccagctggggcacagcgaggcggccccttctcccgacgacgttcgatggagtagggtcccagaccgttgtcccgaagagcgagatcgagcttggccccctccc

SEQ ID NO: 171 - DNA, 255 bp, Homo Sapiens
gaatcgtcgacccacgcgtccgcctccttccatctcatctctatctctgggctggtttgtgagctgaaagagttcacacatacctggggtgggcctctgtctcctcgacagagtgggactgaggagcgaggcctgaagcattactggttcacatcctggcccgaccagaagaccccagaccgggcccccccactcctgcacctggtgcgggaggtggaggaggcagcccagcaggaggggccccactgtgccccc

SEQ ID NO: 172 - DNA, 255 bp, Homo Sapiens
  misc_feature (25)..(25): n is a, c, g, or t
gaatcgtcgacccacgcgtccgccnanacgtncgcccacgctttgcgcgataagtgctcgnccgcctttgtcctgcctactgnatgcnggcgctgngtctgtggggctctggcngnggcctgnccctgggacggacagcatgtnctcgtcccnggaggactggntntgtgcgcnctccatgcacgagttttnngccaaggagatcaacgggcanatggtttaanctgaacaantaccgggnnttctngtgcatng

SEQ ID NO: 173 - DNA, 255 bp, Homo Sapiens
gaatcgtcgacccacgcgtccgggggcgcggcctccggcggaggagggaacagctgggacctcccgggttcccacgtgcggctgccggggcgtgcacagtctgggacccgtggcggtgctggcaacacaagcaccagctgcggggactccaacagcatctgcccggccccctccacgatgtccaaggccgaggaggccaagaagctggcgggccgcgcggctgtggagaaccacgtgaggaataaccaagtgctg

SEQ ID NO: 174 - DNA, 255 bp, Homo Sapiens
gaatcgtcgacccacgcgtccgggaattccagatggtcaaataaaaaaaatgttcctaaacttggtgatatgaattcatcatttgaagatgtagatatattttattctttctggtataattttgattcttggagagaattttcttatttagatgaagaagaaaaagaaaaagcagaatgtcgtgatgagaggagatggattgaaaagcagaacagagcaacaagagcacaaagaaaaaaagaagaaatgaacaga

SEQ ID NO: 175 - DNA, 2983 bp, Homo Sapiens
ggggaggaggccgcgcggggtggggtctggcggtacgcgctggctgcgtcgacgtgctgacgccatgacgccccggctggtgtgtgtcggtgtgtatgtgtgtgtgtgagtgtgcgcgctccgagtgtgtgtgtatttgtgtatcggcggtcccgcaggtcccggatgttgcggacagtatgaggcaagcgcagggggacggggaccagcagctgtcgccgccgctctcaggctctgggaaccacccttctactttctgtctctaggaatttcactactctagggtgaagagggaacagaaatctttgccccctgactttggaaatctcgtttaaccttcaaactggcgatgtcaagggttccaagtcctccacctccggcagaaatgtcgagtggccccgtagctgagagttggtgctacacacagatcaaggtagtgaaattctcctacatgtggaccatcaataactttagcttttgccgggaggaaatgggtgaagtcattaaaagttctacattttcatcaggagcaaatgataaactgaaatggtgtttgcgagtaaaccccaaagggttagatgaagaaagcaaagattacctgtcactttacctgttactggtcagctgtccaaagagtgaagttcgggcaaaattcaaattctccatcctgaatgccaagggagaagaaaccaaagctatggagagtcaacgggcatataggtttgtgcaaggcaaagactggggattcaagaaattcatccgtagagattttcttttggatgaggccaacgggcttctccctgatgacaagcttaccctcttctgcgaggtgagtgttgtgcaagattctgtcaacatttctggccagaataccatgaacatggtaaaggttcctgagtgccggctggcagatgagttaggaggactgtgggagaattcccggttcacagactgctgcttgtgtgttgccggccaggaattccaggctcacaaggctatcttagcagctcgttctccggtttttagtgccatgtttgaacatgaaatggaggagagcaaaaagaatcgagttgaaatcaatgatgtggagcctgaagtttttaaggaaatgatgtgcttcatttacacggggaaggctccaaacctcgacaaaatggctgatgatttgctggcagctgctgacaagtatgccctggagcgcttaaaggtcatgtgtgaggatgccctctgcagtaacctgtccgtggagaacgctgcagaaattctcatcctggccgacctccacagtgcagatcagttgaaaactcaggcagtggatttcatcaactatcatgcttcggatgtcttggagacctctgggtggaagtcaatggtggtgtcacatccccacttggtggctgaggcataccgctctctggcttcagcacagtgcccttttctgggacccccacgcaaacgcctgaagcaatcctaagatcctgcttgttgtaagactccgtttaatttccagaagcagcagccactgttgctgccactgaccaccaggtagacagcgcaatctgtggagcttttactctgttgtgaggggaagagactgcattgtggccccagacttttaaaacagcactaaataacttgggggaaacggggggagggaaaatgaaatgaaaaccctgttgctgcgtcactgtgttccctttggcctggctgagtttgatactgtggggattcagtttaggcgctggcccgaggatatcccagcggtggtacttcggagacacctgtctgcatctgactgagcagaacaaatcgtcaggtgcctggagcaaaaaggaaaaaaaaaaaagaaaggacattgagttttaacagaagggaaaaggaaagaagaaaagatttttgcagaatttctcaaaaatcagtttgtggattccagtagtatttatattgagagaaacaaattttagtccttctaactgtgctaaaacttggatatttgtgaaaactccttaccaccatacaagcatcagaagagctctcttgttgttagcacttattgtttgcaagaacagaatacatccttttatccttttatgaaaaatgacaagtgaaggcaaaaggggaaggttatttgatctggaagatgagtgttctgatgtggtggcttttgcaaaaatctttattggtgttgaaaactggaaaaaataactcatccagaattcatattgtcttgacaagaactatggttctctgtttttagatattgtggaaaatgtttttgggcatttttctctgattttatttcttctcccccacccctttttctaaaaaacaaacaaaaaaaaaaacacacaaaacaaaaacagaacaaaagaagagagaaggaaattttatcaattaaaaatgctgtgtgataaaatcccagcccagattgctcagctgtttgtacctgacttgccgcctgcataggagccagttctgttccttctgactagcccctcttcctccaggggagaacttccaaatgttaatttttttttttttgaaaatataaataattactattttgtactgtgtggtatctctggtcttttgtttcactcacctgccttgtctcttgggtctgagtcccttgcttaagggattttgaagtcctagttttcagcttgcagagattatgtctgaaatgcctaatgagtcgcagggatttgttgagactccgtaatctcaagttctctttgtgagctatcagcatctgccagtctcttgtcctccctgagtatctcacagtccatatcctgatgagggatcaggcccctacctctgccaaggcaagtaatggtagtgggcttttaaactgccccccgtatgttttaagacctaatccccacctcccttcttctaactaaatataaaaagatccaggggacataaatgtggagattaaataaagggaaattattgtctctaaaaaaaaaaaaaaaaa